SEQUENCE LISTING (85 sequences)

SEQ ID NO: 1 - protein, 107 aa, Unknown
  Synthesized
AELVLTQTPSPVSAAVGGTVTINCQSSRSVYSQNRLSWYQHKPGQPPKLLVYAASTLPSGVPSRFKGSGSGTQFTLTISDVQCDDAATYYCAGAYSGDSVFGGGTEL

SEQ ID NO: 2 - protein, 114 aa, Unknown
  Synthesized
QSLEESGGRLVTPGTPLTLTCTVSGFTISSGDMSWVRQAPGKGLEWIGIIYASGNTAYASWAKGRFTISRTSTTVDLKMASPTTEDTATYFCARGNPGWGSVVWGPGTLVTVSS

SEQ ID NO: 3 - protein, 103 aa, Unknown
  Synthesized
AELALTQPPSVSGSPGQSITISCTGGGHYNYLSWYQQHPGKAPRLILYAVYNRPSGVSHRFSGSKSGTTASLTISGLQAEDEADYYCAGAYSGDSVFGGGTKL

SEQ ID NO: 4 - protein, 116 aa, Unknown
  Synthesized
VQLVESGGGLVQPGGSLTLSCAASGFTVSSGDMSWVRQAPGKGLEWIGIIYASGNTAYASSAKGRFTISRDNSRNTVSLQMNSLRAEDTAVYYCARGNPGWGSVVWGQGTLVTVSS

SEQ ID NO: 5 - DNA, 37 bp, Unknown
  Synthesized
gggcccaggcggccgagctccagatgacccagtctcc

SEQ ID NO: 6 - DNA, 37 bp, Unknown
  Synthesized
gggcccaggcggccgagctcgtgatgacycagtctcc

SEQ ID NO: 7 - DNA, 37 bp, Unknown
  Synthesized
gggcccaggcggccgagctcgtgwtgacrcagtctcc

SEQ ID NO: 8 - DNA, 37 bp, Unknown
  Synthesized
gggcccaggcggccgagctcacactcacgcagtctcc

SEQ ID NO: 9 - DNA, 40 bp, Unknown
  Synthesized
gggcccaggcggccgagctcgtgbtgacgcagccgccctc

SEQ ID NO: 10 - DNA, 40 bp, Unknown
  Synthesized
gggcccaggcggccgagctcgtgctgactcagccaccctc

SEQ ID NO: 11 - DNA, 43 bp, Unknown
  Synthesized
gggcccaggcggccgagctcgccctgactcagcctccctccgt

SEQ ID NO: 12 - DNA, 46 bp, Unknown
  Synthesized
gggcccaggcggccgagctcgagctgactcagccaccctcagtgtc

SEQ ID NO: 13 - DNA, 40 bp, Unknown
  Synthesized
gggcccaggcggccgagctcgtgctgactcaatcgccctc

SEQ ID NO: 14 - DNA, 40 bp, Unknown
  Synthesized
gggcccaggcggccgagctcatgctgactcagccccactc

SEQ ID NO: 15 - DNA, 40 bp, Unknown
  Synthesized
gggcccaggcggccgagctcgggcagactcagcagctctc

SEQ ID NO: 16 - DNA, 40 bp, Unknown
  Synthesized
gggcccaggcggccgagctcgtggtgacycaggagccmtc

SEQ ID NO: 17 - DNA, 40 bp, Unknown
  Synthesized
gggcccaggcggccgagctcgtgctgactcagccaccttc

SEQ ID NO: 18 - DNA, 23 bp, Unknown
  Synthesized
cagtaatacactgcaaaatcttc

SEQ ID NO: 19 - DNA, 23 bp, Unknown
  Synthesized
cagtaataaaccccaacatcctc

SEQ ID NO: 20 - DNA, 23 bp, Unknown
  Synthesized
cagtaataagttgcgaaatcatc

SEQ ID NO: 21 - DNA, 21 bp, Unknown
  Synthesized
gcagtaataatcagcctcrtc

SEQ ID NO: 22 - DNA, 20 bp, Unknown
  Synthesized
cagtaataatcagcctcrtc

SEQ ID NO: 23 - DNA, 72 bp, Unknown
  Synthesized
gaagattttgcagtgtattactgcgcaggcgcttatagtggtgatagtgtttttggccaggggaccaagctg

SEQ ID NO: 24 - DNA, 72 bp, Unknown
  Synthesized
gaggatgttggggtttattactgcgcaggcgcttatagtggtgatagtgtttttggccaggggaccaagctg

SEQ ID NO: 25 - DNA, 72 bp, Unknown
  Synthesized
gatgatttcgcaacttattactgcgcaggcgcttatagtggtgatagtgtttttggccaggggaccaagctg

SEQ ID NO: 26 - DNA, 69 bp, Unknown
  Synthesized
gaygaggctgattattactgcgcaggcgcttatagtggtgatagtgttttcggcggagggaccaagctg

SEQ ID NO: 27 - DNA, 44 bp, Unknown
  Synthesized
ggtggttcctctagatcttcccaggtgcagctggtgcagtctgg

SEQ ID NO: 28 - DNA, 44 bp, Unknown
  Synthesized
ggtggttcctctagatcttcccagatcaccttgaaggagtctgg

SEQ ID NO: 29 - DNA, 44 bp, Unknown
  Synthesized
ggtggttcctctagatcttccgaggtgcagctggtgsagtctgg

SEQ ID NO: 30 - DNA, 43 bp, Unknown
  Synthesized
ggtggttcctctagatcttccgaggtgcagctgktggagtctg

SEQ ID NO: 31 - DNA, 44 bp, Unknown
  Synthesized
ggtggttcctctagatcttcccaggtgcagctacagcagtgggg

SEQ ID NO: 32 - DNA, 44 bp, Unknown
  Synthesized
ggtggttcctctagatcttcccaggtgcagctgcaggagtcggg

SEQ ID NO: 33 - DNA, 72 bp, Unknown
  Synthesized
gacacggccgtgtattactgtgcgcgtgggaatcctggttggggtagtgtcgtctggggccagggaaccctg

SEQ ID NO: 34 - DNA, 24 bp, Unknown
  Synthesized
cgcacagtaatacacggccgtgtc

SEQ ID NO: 35 - DNA, 65 bp, Unknown
  Synthesized
gcactgctcgcgtatgcagtgttaccactagcgtaaatgattccaatccactccagccccttccc

SEQ ID NO: 36 - DNA, 27 bp, Unknown
  Synthesized
cactgcatacgcgagcagtgcaaaagg

SEQ ID NO: 37 - DNA, 38 bp, Unknown
  Synthesized
gggcccaggcggccgagctcgtgmtgacccagactcca

SEQ ID NO: 38 - DNA, 42 bp, Unknown
  Synthesized
ggaagatctagaggaaccacctaggatctccagctcggtccc

SEQ ID NO: 39 - DNA, 42 bp, Unknown
  Synthesized
ggtggttcctctagatcttcccagtcgytggaggagtccggg

SEQ ID NO: 40 - DNA, 46 bp, Unknown
  Synthesized
cctggccggcctggccactagtgaccgatgggcccttggtggargc

SEQ ID NO: 41 - DNA, 41 bp, Unknown
  Synthesized
gaggaggaggaggaggaggcggggcccaggcggccgagctc

SEQ ID NO: 42 - DNA, 41 bp, Unknown
  Synthesized
gaggaggaggaggaggagcctggccggcctggccactagtg

SEQ ID NO: 43 - DNA, 40 bp, Unknown
  Synthesized
gaggaggaggaggaggctagcatgaaggtctccgcggcac

SEQ ID NO: 44 - DNA, 62 bp, Unknown
  Synthesized
ggaacgtcgtacgggtactggccggcctggccgctcatctccaaagagttgatgtactcccg

SEQ ID NO: 45 - DNA, 44 bp, Unknown
  Synthesized
gcggaggagcttgctagctgcgaggggcccaggcggccgagctc

SEQ ID NO: 46 - DNA, 24 bp, Unknown
  Synthesized
gcggaggagcttgctagctgcgag

SEQ ID NO: 47 - skipped
  Synthesized

SEQ ID NO: 48 - DNA, 21 bp, Unknown
  Synthesized
ggtggttcctctagatcttcc

SEQ ID NO: 49 - skipped
  Synthesized

SEQ ID NO: 50 - DNA, 45 bp, Unknown
  Synthesized
ggaagatctagaggaaccaccgcctaggacggtcascttggtscc

SEQ ID NO: 51 - DNA, 42 bp, Unknown
  Synthesized
ggaagatctagaggaaccacctttgatctccagcttggtccc

SEQ ID NO: 52 - DNA, 22 bp, Unknown
  Synthesized
aagacagctatcgcgattgcag

SEQ ID NO: 53 - DNA, 24 bp, Unknown
  Synthesized
gcccccttattagcgtttgccatc

SEQ ID NO: 54 - DNA, 22 bp, Unknown
  Synthesized
ctattgcctacggcagccgctg

SEQ ID NO: 55 - protein, 24 aa, Unknown
  Synthesized
YTSEYTSEYTSEYTSQYTSQYTSQ

SEQ ID NO: 56 - skipped
  Synthesized

SEQ ID NO: 57 - DNA, 47 bp, Unknown
  Synthesized
gaggaggaggaggaggagctcactcccagtcgttggaggagtccggg

SEQ ID NO: 58 - DNA, 47 bp, Unknown
  Synthesized
gaggaggaggaggaggagctcactccgaggtgcagctggtggagtct

SEQ ID NO: 59 - DNA, 44 bp, Unknown
  Synthesized
gctgccaggtgccagatgtgccgagatcgtgctgacccagactc

SEQ ID NO: 60 - DNA, 48 bp, Unknown
  Synthesized
gctgccaggtgccagatgtgcccagtctgccctgactcagcctccctc

SEQ ID NO: 61 - DNA, 46 bp, Unknown
  Synthesized
gaggaggaggaggagactagtgctctggctgccaggtgccagatgt

SEQ ID NO: 62 - protein, 7 aa, Unknown
  Synthesized
GGSSRSS

SEQ ID NO: 63 - protein, 4 aa, Unknown
  Synthesized
KDEL

SEQ ID NO: 64 - protein, 9 aa, Unknown
  Synthesized
YPYDVPDYA

SEQ ID NO: 65 - protein, 4 aa, Unknown
  Synthesized
YTSE

SEQ ID NO: 66 - protein, 4 aa, Unknown
  Synthesized
YTSQ

SEQ ID NO: 67 - protein, 107 aa, Unknown
  Synthesized
AELELTQPPSVSGSPGQSITISCTVTSSDVGSYNFVSWYQQHPGKAPKLIIYDATRRPSGVSNRFSGSKSGDTASLTISGLRAEDEADYYCAGAYSGDSVFGGGTKL

SEQ ID NO: 68 - protein, 107 aa, Unknown
  Synthesized
AELALTQPPSVSGSPGQSITIPCTGTSSDVGGYNFVSWYQQRPGNAPKLILYGVTKRPSGISDRFSGSKSGNVASLTISGLQPDDEADYYCAGAYSGDSVFGGGTKL

SEQ ID NO: 69 - protein, 106 aa, Unknown
  Synthesized
AELTLTQSPGTLSLSPGERATLSCRASQTLSGNRLAWYQQKPGQAPRLLIYGASRRASGIPDRISGSGSGTDFTLTISRLDPEDFAVYYCAGAYSGDSVFGQGTKL

SEQ ID NO: 70 - protein, 33 aa, Unknown
  Synthesized
MDYQVSSPIYDINYYTSEPCQKINVKQIAARLL

SEQ ID NO: 71 - protein, 12 aa, Unknown
  Synthesized
HAPWSLITYTSE

SEQ ID NO: 72 - protein, 12 aa, Unknown
  Synthesized
FKLDAYTSQFLI

SEQ ID NO: 73 - protein, 11 aa, Unknown
  Synthesized
MDYQVSSPIYD

SEQ ID NO: 74 - protein, 11 aa, Unknown
  Synthesized
SPIYDINYYTS

SEQ ID NO: 75 - protein, 11 aa, Unknown
  Synthesized
NYYTSEPCQKI

SEQ ID NO: 76 - protein, 11 aa, Unknown
  Synthesized
PCQKINVKQIA

SEQ ID NO: 77 - protein, 9 aa, Unknown
  Synthesized
VKQIAARLL

SEQ ID NO: 78 - protein, 4 aa, Unknown
  Synthesized
YTSD

SEQ ID NO: 79 - protein, 6 aa, Unknown
  Synthesized
YYTSEP

SEQ ID NO: 80 - protein, 7 aa, Unknown
  Synthesized; Fusion with MBP polypeptide
YYTSEPG

SEQ ID NO: 81 - protein, 5 aa, Unknown
  Synthesized; Fusion with MBP polypeptide
YTSEG

SEQ ID NO: 82 - protein, 4 aa, Unknown
  Synthesized; Fusion with MBP polypeptide
YTSG

SEQ ID NO: 83 - protein, 5 aa, Unknown
  Synthesized; Fusion with MBP polypeptide
YTSDG

SEQ ID NO: 84 - protein, 5 aa, Unknown
  Synthesized; Fusion with MBP polypeptide
YTSQG

SEQ ID NO: 85 - protein, 6 aa, Unknown
  Synthesized; Fusion with MBP polypeptide
YYTSEP

(19) United States
(12) Patent Application Publication
(10) Pub. No.: US 2003/0049251 A1
(43) Pub. Date: Mar. 13, 2003

(54) Methods and compositions useful for inhibiting ccr5-dependent infection of cells by hiv-1

(76) Inventors: Carlos F. Barbas, Solana Beach, CA (US); Peter Steinberger, Wein (AT)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(21) Appl. No.: 09/913,238

(22) PCT Filed: Dec. 8, 2000
(86) PCT No.: PCT/EP00/12419

(51) Int. Cl. A61K 39/395; C07K 16/28

(52) U.S. Cl. 424/144.1; 530/388.1; 424/142.1

(57) ABSTRACT

A method for inhibiting, diminishing, preventing or treating pathogenic infection of cells comprising expressing a recombinant antibody protein fused to an intracellular anchor means, wherein said antibody is specific for a surface receptor of said cells necessary for pathogenic infection, wherein said antibody is suitably selected from CCR5 and CXCR4 specific antibodies. A recombinant antibody protein fused to an intracellular anchor means which is specific for a surface receptor necessary for pathogenic infection, but suitably elected from CCR5 and CXCR4 specific antibodies and humanized antibodies therefrom. A recombinant antibody that immunoreacts with CCR5 or CXCR4 surface receptor. Peptides comprising at least YTSF or YTSQ sequence for use in a vaccine or an immunogenic composition intended to control, prevent, diminish or treat HIV infections. An antiidiotypic antibody mimicking CCR5 or CXCR4 epitopes raised from anti-CCR5 and anti-CXCR4 antibodies.

The invention relates to compositions and methods for inhibiting pathogenic infection of cells where infection is surface receptor-dependent, and particularly to antibody constructs which immunoreact with surface receptors, in particular human CCR5 and CXCR4, and which are useful for interfering with the ability of surface receptors to interact with pathogens.

### BACKGROUND

The molecular mechanism of human immunodeficiency virus type 1 (HIV-1) entry into cells involves specific interactions between the viral envelope glycoproteins (env) and two target cell proteins, CD4 and a chemokine receptor. HIV-1 cell tropism is determined by the specificity of the env for a particular chemokine receptor. Macrophage (M)- tropic viruses require CCR5 for entry and these viruses are designated as R5 viruses. T-cell line (TCL)- tropic viruses use CXCR4 for entry and are designated as X4 viruses (Berger et al. Nature 391- 240, 1998). While a multiplicity of coreceptors have been shown to facilitate HIV-1 entry in vitro, only CCR5 and CXCR4 have been convincingly demonstrated to be relevant in vivo (Berger et al., Annu Rev Immunol, 17: 657-700, 1999; Zhang et al., J Virol, 73:3443-3448, 1999).

Several findings suggest that CCR5-positive cells are typically the critical first targets in HIV-1 infection and that CCR5 expression levels are key in disease progression. Individuals with a homozygous deletion (Villinger et al., Immunol Lett, 66: 37-46, 1999) in their CCR5 gene lack functional CCR5 expression and are highly protected against transmission which usually involves R5 viruses (Berger, 1999 supra). Individuals that are heterozygous for this mutation express reduced levels of CCR5 and are delayed in their progression to AIDS by 1-2 years (Dean et al., Science, 73: 1856-1862, 1999). Furthermore, the 59029 G/A polymorphism reduces the activity of the CCR5 promoter by ˜45%; individuals with this mutation are delayed in their progression to AIDS by ˜4 years (McDermott et al., Lancet, 352: 866-870, 1998). Significantly, these natural polymorphisms are not known to be associated with any detrimental phenotype. Therefore, intervention strategies aimed at blocking CCR5 expression should be beneficial for cellular protection against viral infection and may provide a clinical benefit.

In attempts to disrupt HIV-1 replication, intracellular immunization strategies based on the expression of trans-dominant mutants, ribozymes and intracellular antibodies (intrabodies) have been studied (Malim et al., Science, 247: 1222-1225, 1990; Marasco et al., PNAS, 90: 7889-7893, 1993).

Approaches that aim to prevent viral entry should have advantages over strategies that target post-entry steps of the HIV-1 life cycle. In this direction, intracellular expression of chemokines has shown some promise in limiting, to some extent, viral entry (Yang et al., PNAS, 94: 11567-11572, 1997; Yang et al., Hum Gene Ther., 9: 2005-2018, 1998; Chen et al., Nat Med, 3: 1110-1116, 1997).

Mice have been the main source of monoclonal antibodies for the past decades. Two advents have recently made rabbit which are widely used for the production of polyclonal antibodies an interesting alternative source of monoclonal antibodies: The finding of a fusion partner for rabbit B-cells (Spieker-Polet et al., PNAS, 92(20): 9348-52, 1995) and phage display of antibody fragments derived from immune rabbits (Lang et al., Gene, 172(2): 295-298, 1996; Ridder et al., Biotechnology, 13(3): 255-60, 1995). As with mouse monoclonal antibodies, the use of rabbit antibodies with therapeutic potential is limited through their immunogenicity and requires humanization. A variety of humanization procedures have been reported for mouse mAb. Many of them are based on grafting of CDRs onto human frameworks. In most cases this leads to a considerable loss of affinity and in extreme cases there is no specific binding to the antigen detectable (Baca et al., J Biol Chem, 72(16): 10678-84, 1997). Therefore in most cases, CDR-grafting is combined by changes in framework residues that are potentially important for binding to generate humanized antibodies that have similar affinities as the parental antibodies. It is believed that the constraints of humanization of rabbit antibodies are similar to the ones observed with murine antibodies. A methodology based on a combination of CDR grafting and selection of V sequences from human antibody repertoire has been successfully employed to humanize a murine antibody specific to human avb3 integrin (Rader et al., PNAS, 95(15): 8910-5, 1998). The authors preserved the original CDR3 (L and H) that are considered to generally make the most significant contribution to affinity and specificity.

Approaches aiming to prevent viral entry of the HIV-1 by use of extracellular CCR5 and/or CXCR4 specific antibodies have been reported in WO00/55207 (Berger et al.), WO 00/53633 (Mack et al.), WO 00/50088 (Millennium Pharma), WO 00/40964 (US Dep. Health) and WO 00/35409 (Progenics Pharma), Olson et al. (J. of Virology, 4145-4155, 1999), Chang et al. (P.N.A.S., 96:10367-10372, 1999), and T. Lehner et al. (Eur. J. Immunol., 29:2427-2435, 1999), for instance. An important question facing extracellular CCR5 targeted strategies is if they will encourage a phenotypic switch to the more virulent X4 virus (Michael et al., Nat Med., 5: 740-2, 1999) which uses CXCR4 as a co-receptor, a shift that is characterized by a syncytium-inducing phenotype and accelerated destruction of CD4⁺ T-cells (Berger, 1999 supra).

Today it has not been reported any strategy using intracellular expression of antibodies, or functional fragments thereof, to functionally delete transit of a surface receptor necessary for pathogen entry. There is also a need to further define immunogenic means capable of promoting control and regulation of CCR5 or CXCR4 interactions with a ligand.

### SUMMARY OF THE INVENTION

Therefore, the present invention aims to cover a method for inhibiting, diminishing, preventing or treating pathogenic infection of cells comprising expressing a recombinant antibody protein fused to an intracellular anchor means, wherein said antibody is specific for a surface receptor of said cells necessary for said pathogenic infection.

The invention also relates to a recombinant antibody protein fused to an intracellular anchor means which is specific for a surface receptor necessary for pathogenic infection, particularly antibody selected from CCR5 and CXCR4 specific antibodies.

A further aspect relates to a recombinant antibody that immunoreacts with CCR5 or CXCR4.

Another aspect relates to polynucleotide that encodes an antibody according to the invention, and to viral expression systems encoding a polynucleotide of the invention.

Another aspect relates to peptides comprising at least YTSE or YTSQ amino acid sequence for use in a vaccine or an immunogenic composition intended to control, prevent, diminish or treat HIV infections.

In a related aspect, the invention relates to anti-idiotypic antibody mimicking CCR5 or CXCR4 epitopes raised from anti-CCR5 and CXCR4 antibodies.

### DESCRIPTION OF THE INVENTION

The practice of the present invention will employ, unless otherwise indicated conventional techniques of cell biology, molecular biology, cell culture, immunology, virology, and the like which are in the skill of one in the art. These techniques are fully disclosed in the current literature and reference is made specifically to Sambrook, Fritsch and Maniatis eds., “Molecular Cloning, A Laboratory Manual”, 2^{nd} Ed., Cold Springs Harbor Laboratory Press, (1989); Celis, J. E. “Cell Biology, A Laboratory Handbook”, Academic Press, Inc. (1994); Coligan et al., “Current Protocols in Immunology”, John Wiley and Sons (1991); and Harlow et al., “Antibodies: A Laboratory Manual” (1988), Biosupplynet Source Book (1999), Cold Springs Harbor Laboratory. All publications and patent applications cited in this specification are indicative of the level of skill of those skilled in the art to which this invention pertains, and are hereby incorporated by reference in their entirety.

Abbreviations used in the following specification are: HIV-1, human immunodeficiency virus type 1; V_{H}, heavy-chain variable region; V_{L}, light chain variable region; scFv, single chain antibody fragment; HSA, heat stable antigen; env, viral envelope glycoprotein; SIV, simian immunodeficiency virus; FR, framework region; CDR complementary determining region; HFR, heavy chain framework region; HCDR, heavy chain complementary determining region; LFR, light chain framework region; LCDR, light chain complementary determining region; mAb, monoclonal antibodies.

An “intrabody” designates an antibody which has been fused with intracellular anchor mean so that it remains in the intracellular region.

The invention describes generally compositions and methods for inhibiting, diminishing, preventing or treating surface receptor-dependent infection of cells by pathogenic agents, particularly from virus, bacteria, or eucaryotes parasites, for instance, such as human respiratory syncytial virus (I. Martinez et al., J. G. Virology, 81:2715-2722, 2000), Influenzy virus (U.S. Pat. No. 6,150,131), cytomegalovirus, hepatitis B virus, human papillomavirus, Epstein-Barr virus, human herpes virus, human immunodeficiency virus (HIV), Mycoplasma penetrans, Staphylococcus aureus, streptococcus pneumoniae, Poliovirus, Parvoviruses, and malaria, for instance.

Surprisingly it has been demonstrated that pathogenic interactions with cells, like HIV env interactions with CCR5-bearing cells, do not block or compete with immunoreactions between antibodies of the invention and surface receptors targeted by these antibodies.

More particularly, the ST6 and ST6/34 intrabodies of the invention have been shown to be unexpectively superior to RANTES-intrakine in blocking CCR5 surface expression and in preventing cell-cell fusion events. The efficiency of the ST6 and ST6/34 intrabodies in terms of functional deletion of the coreceptor CCR5 is evident in the resistance it imparts to cells in the face of stringent viral and infected cell challenge. Extended in vitro challenge of a receptor deleted cell line with infected cells resulted, in time, in a culture consisting of virtually only the receptor deleted HIV-1-resistant cell line. This outcome can be obviously repeated in vivo, allowing likely for the establishment of an HIV-1-resistant cell pool in infected individuals.

According to a first aspect, the invention covers a method for inhibiting, diminishing, preventing or treating pathogenic infection of cells comprising expressing a recombinant antibody protein fused to an intracellular anchor means, wherein said antibody is specific for a surface receptor of said cells necessary for pathogenic infection. In particular, the invention contemplates methods for inhibiting surface receptor dependent infections, practiced ex vivo or in vivo, as described further herein.

The recombinant antibody construct is thus contacted with surface receptor protein intracellularly, thereby interfering with receptor function before it has an opportunity to be transported to the cell surface where it can interact with infectious pathogenic agents. A fusion protein having an intracellular anchor means is particularly essential for practicing the present invention. When present in a cell, the anti-surface receptor domain of the fusion protein immunoreacts with and binds to any expressed surface receptor protein in the intracellular regions of the cell in the form of an immunoreaction complex. Thereafter, the anchor means functions to retain the immunoreaction complex within the cell, and thereby prevent the expressed surface receptor from transit, e.g. becoming processed and expressed on the surface of cells.

To that end, the invention comprises expressing a vector inside a cell that expresses surface receptors, thereby providing an intracellular supply of anti-surface receptor antibody construct to the cell, which construct in turn immunoreacts with any surface receptor present inside the cell to form an immunoreaction complex.

The vector can be any of a variety of vectors which express an antibody construct of this invention, as are well known in the art. Such vectors can be designed in the form of plasmids, viruses or other engineered constructs for introducing nucleotide sequences into cells. The vectors can be introduced as transient or stable entities in the cell for short or long term expression of the antibody construct, depending on the desired application. Methods for introducing (i.e, transforming or transfecting) nucleic acids into a cell can vary widely, as is well known, and therefore the invention need not be so limited. Exemplary vectors for stable introduction comprise retroviral vectors. Additional vectors include the lentiviral-based, adenovirus-based, AAV-based gene transfer vectors. Vectors described below are particularly suitable for that purpose.

The method for expressing a vector that encodes a fusion antibody of this invention into a surface receptor-bearing cell, comprises introducing and maintaining the cell for a time period sufficient for the vector to express the encoded protein. In one embodiment, the promoters controlling expression of the fusion protein are inducible, thereby allowing external control of the timing of expression of the encoded fusion protein. In a most preferred embodiment, the recombinant antibody used is selected from CCR5 and CXCR4 specific antibodies, which are receptors necessary for HIV entry into T cells, such as ST6 or ST6/34. These antibodies can be co-expressed, on the same vector or on two separate vectors, as described further in the examples.

Since ST6 and ST6/34 react with CCR5 from non-human primates, this strategy can be tested in SIV and SHIV models of human AIDS allowing the benefits as well as the potential drawbacks of this approach to be assessed. Results of the following examples indicate clearly that the introduction of a surface receptor specific intrabody into stem cells, particularly CCR5 and/or CXCR4 specific intrabody into hematopoietic stem cells, is a suitable strategy for the generation of a cell pool in infected individuals that is protected from HIV-1 infection, particularly R5- HIV-1 infection.

Other antibodies may be however used suitably against cell-surface receptors necessary for pathogenicity of pathogenic agent, such as, but not limited to, respiratory syncytial virus, Influenzy virus, cytomegalovirus, hepatitis B virus, human papillomavirus, Epstein-Barr virus, human herpes virus, Mycoplasma penetrans, Staphylococcus aureus, Streptococcus pneumoniae, Poliovirus, Parvoviruses, and malaria, for instance.

An “Antibody” refers to a protein consisting of one or more polypeptides substantially encoded by immunoglobulin genes. It may encompass the intrabody of the invention. The recognized immunoglobulin genes may include the kappa, lambda, alpha, gamma, delta, epsilon and mu constant region genes, but will contain at least the hypervariable loops or CDRs which determine tropism to ligands. The antibodies may exist in a variety of forms, including, but not limited to, Fv, Fab, and F(ab)2, as well as in single chains (scFv).

scFv molecules consist of domains (VL and VH) of the same nature. The findings as illustrated in the examples unexpectedly show that such molecules can be produced efficiently and is proven functional for all its components. Surprisingly, dimerization of each chain, i.e. VL and VH, can be better achieved with peptide linkers as short as a few amino acids, but preferably less than 10 residues, for instance consisting of 7 residues or less, such as GGSSRSS. Preferred scFv-fusion proteins are those comprising a scFv domain that immunoreacts with CCR5 or CXCR4 fused to an intracellular anchor means, in particular scFv domain providing either the VL and/or VH of ST6 (respectively SEQ ID NO:1 and NO:2), or the VL and/or VH of ST6/34 (respectively SEQ ID NO:3 and NO:4).

An intracellular anchor means is any sequence of amino acid residues which when present in the fusion protein provides the capacity be retained inside the cell rather than allow the fusion protein, upon expression to be processed and expressed on the cell surface. Preferably, the intracellular anchor means is a sequence of amino acids which when present in the fusion protein provides the capacity to interact with the cell's endoplasmic reticulum (ER) and thereby be restricted in cellular transport such that the fusion protein is sequestered in the ER milieu and not transported to the cell surface. This intracellular anchor means can be fused at the C terminus of at least one antibody chain, i.e. VL and/or VH. An exemplary intracellular anchor means is an ER retention peptide domain. A preferred ER retention peptide domain has the amino acid residue sequence lysine-aspartic acid-glutamic acid-leucine (KDEL). Other suitable retention peptides are described in Skalnik et al. (J. Biol. Chem., 263:6836-41, 1988), Kwon et al. (FEBS Let., 475:27-30, 2000), Laplante et al. (Biochem J., 348:189-99, 2000), Hubbart M J et al. (Eur J. Biochem, 267:1945-57, 2000), Yamagouchi et al. (J. Cell Biol., 147:1195-204, 1999) and Bassuk et al. (Matrix, 9:244-58, 1989), for instance. Other exemplars of intracellular anchor means are peptides that enable localization to Golgi region, or polypeptides having specific affinity to intracellular proteins, for instance. One example is the carboxyterminal 30 amino acids of GLUT4 that regulate intracellular localization (see EP721508). Inducible intracellular anchor means is preferred, either induced by promoter inducibility or by other external factors, such as insuline in case of carboxyterminal 30 amino acids of GLUT4.

The target cells of the invention are mammalian cells and these include but are not limited to humans, mice, monkeys, chimpanzees, farm animals; such as cattle, sheep, pigs, goats, and horses, sport animals, pets; such as dogs and cats, and other laboratory rodents and animals; such as mice, rats, guinea pigs and the like. Preferably the target cells are human cells. Preferred human cells include liver, hematopoietic, smooth muscle, neural, endothelial vascular cells, tumor cells and epithelial cells. Hematopoietic cells are particularly preferred, and these cells encompass hematopoietic stem cells, erythrocytes, neutrophils, monocytes, platelets, mast cells, eosinophils and basophils, B and T lymphocytes, dentritic cells and NK cells as well as the respective lineage progenitor cells. Hematopoietic stem cells and T-cells are especially preferred. Hematopoietic stem cells (HSC) are defined as a population of hematopoietic cells containing long term multilineage repopulating potential. T-cells are defined as a type of lymphocyte and are thought to develop from hematopoietic stem cells. There are many types of T-cells including cytotoxic T-cells, helper T-cells, inducer T-cells and supressor T cells.

Methods of obtaining target cells, particularly hematopoietic cells are well known in the art and not repeated herein. Non-limiting sources of hematopoietic cells, including hematopoietic stem cells, are bone marrow, embryonic yolk sac, fetal liver tissue, adult spleen, and blood such as adult peripheral blood and umbilical cord blood. (To et al., Blood 89:2233,1997). Bone marrow cells may be obtained from ilium, sternum, tibiae, femora, spine and other bone cavities.

The manner in which target cells may be separated from other cells is not critical to this invention. Various procedures may be employed and include physical separation, magnetic separation using antibody-coated magnetic beads, affinity chromatography, and cytotoxic agents joined to a monoclonal antibody or used in conjunction with a monoclonal antibody. Also included is the use of fluorescence activated cell sorters (FACS) wherein the cells can be separated on the basis of the level of staining of the particular antigens. These techniques are well known to those skilled in the art and are described in various references including U.S. Pat. Nos. 5,061,620; 5,409,8213; 5,677,136; and 5,750,397; and Yau et al. (Exp. Hematol., 18:219-222,1990).

The order of cell separation is not critical to the invention, and specific cell types may be separated either prior to genetic modification with the mutated PTKR or after genetic modification. Preferably cells are initially separated by a coarse separation followed by using positive and/or negative selection. In humans the surface antigen expression profile of an enriched hematopoietic stem cell population may be identified by CD34⁺Thy-1⁺Lin⁻. Other nonlimiting enriched phenotypes may include: CD2⁻, CD3⁻, CD4⁻, CD8⁻, CD10⁻, CD14⁻, CD15⁻, CD19⁻, CD20⁻, CD33⁻, CD34⁻, CD38^{1o/-}, CD45, CD59^{+/-}, CD71⁻, CDW109⁺, glycophorin⁻, AC133⁺, HLA-DR^{+/-}, and EM⁺. Lin⁻ refers to a cell population selected on the basis of lack of expression of at least one lineage specific marker, such as, CD2, CD3, CD14, CD15 and CD56. The combination of expression markers used to isolate and define an enriched HSC population may vary depending on various factors and may vary as other express markers become available.

Murine HSCs with similar properties to the human CD34⁺Thy-1⁺Lin⁻ may be identified by kit⁺Thy-1.1^{lo}Lin^{-/lo}Sca-1⁺ (KTLS). Other phenotypes are well known. When CD34 expression is combined with selection for Thy-1, a composition comprising approximately fewer than 5% lineage committed cells can be isolated (U.S. Pat. No. 5,061,620).

It has been shown that CD3 is expressed on most T cells, and that these cells can express the cell surface antigens CD2, CD4, and CD8 antigens. Also CD45 is a useful T cell marker. The most well known T cell marker is the T cell antigen receptor (TCR). There are presently two defined types of TCRs; α, β- TCR and γ, δ- TCR. B cells may be selected, for example, by expression of CD19 and CD20. Myeloid cells may be selected for example, by expression of CD14, CD15 and CD16. NK cells may be selected based on expression of CD56 and CD16. Erythrocytes may be identified by expression of glycophorin A. Neuronal cells may be identified by NCAM and LNGFR (Baldwin et al., J. Cell Biochem.,15:502,1996). Vascular endothelial cells may be identified by VEGFR2, CD34, P-Selectin, VCAM-1, ELAM-1, and ICAM-1.(Horvathova et al., Biol. Trace Elem. Res., 69: 15-26,1999). One skilled in the art is aware of other useful markers for the identification of other target cells.

Once a population containing the target cells are harvested and target cells, particularly hematopoietic cells, are separated, the cells are cultured in a suitable medium comprising a combination of growth factors that are sufficient to maintain growth.

Methods for culturing target cells are well known to those skilled in the art, and these methods are only briefly mentioned herein. Any suitable culture container may be used, and these are readily available from commercial vendors. The seeding level is not critical and will depend on the type of cells used, but in general the seeding level for hematopoietic cells will be at least 10 cells per ml, more usually at least about 100 cells per ml and generally not more than 10⁶ cells per ml when the cells express CD34. Various culture media, solid or liquid, can be used and non-limiting examples include DMEM, IMDM, X-vivo 15 and RPMI-1640. These are commercially available from various vendors. The formulations may be supplemented with a variety of different nutrients, growth factors, such as cytokines and the like. The medium can be serum free or supplemented with suitable amounts of serum such as fetal calf serum, autologous serum or plasma. If cells or cellular products are to be used in humans, the medium will preferably be serum free or supplemented with autologous serum or plasma. (Lansdorp et al., J. Exp. Med., 175:1501,1992) and Petzer, et al. (PNAS, 93:1470, 1996). Also reference is made to Freshney, R. I., “Culture of Animal Cells, A Manual of Basic Techniques”, Wiley-Liss, Inc. (1994).

Non-limiting examples of compounds which may be used to supplement the culture medium are TPO, FL, KL, IL-1, IL-2, IL-3, IL-6, IL- 12, IL-11, stem cell factor, G-CSF, GM-CSF, Stl factor, MCGF, LIF MIP-1α and EPO. These compounds may be used alone or in any combination, and preferred concentration ranges may be readily determined from the published art. When murine stem cells are cultured, a preferred non-limiting medium includes mIL-3, mIL-6 and mSCF. Other molecules can be added to the culture media, for instance, adhesion molecules, such as fibronection or RetroNectin™ (Takara Shuzo Co., Japan).

In vitro systems for measurement of mammalian stem cell activity include the long-term culture initiating cell assay (LTCIC) and the cobblestone-area-forming cell (CAFC) assay. See for instance Pettengell et al. (Blood,84:3653,1994), Breems et al. (Leukemia 8:1095,1994), Reading et al. (Exp. Hem., 22:786, Abst # 406,1994), and Ploemacher et al. (Blood, 74:2755,1989). In the CAFC assay a sparsely plated cell population is simply tested for its ability to form distinct clonal outgrowths (or cobblestone areas) on a stromal cell monolayer over a period of time. This assay gives frequency readouts that correlate with LTCIC and are predictive of engraftment in in vivo assays and patients. A particularly preferred CAFC assay is described in Young et al. (Blood, 88:1619,1996). Flow cytometry can be used to subset hematopoietic cells from various tissue sources by the surface antigens they express. A combination of these assays may be used to test for hematopoietic cells or stem cells.

In a further embodiment, the antibody used in the present method is humanized, i.e. genetically engineered and assembled to retain as little as possible varying amount of the non-human antibody protein sequence in order to avoid immune response when expressed in, or administred to, a patient. Humanized forms of non-human (e.g., murine or rabbit) antibodies are chimeric immunoglobulins, immunoglobulin chains or fragments thereof (such as Fv, Fab, Fab′, F(ab′)2or other antigen-binding sequences regions). Humanized antibodies include human antibodies in which residues from a complementary determining region (CDR) of the human antibody are replaced by residues from a CDR of a non-human species such as mouse, rat or rabbit. In most cases, the humanized antibody comprises variable region domains, in which all or substantially all of the CDR regions are of non-human origin. In some instances, Fv framework region (FR) residues of the human immunoglobulin are replaced by corresponding non- human residues. In order to find utility in the methods of the invention, humanized antibodies must maintain high affinity for the peptide antigen and other favorable biological properties. Methods for humanizing non-human antibodies are well known in the art, including the one described in example 2 herein under. The following methods are well know and can be suitably used or adapted for this purpose: EP125023 EP120694, EP526953, WO9311794, EP549581, EP519596, EP239400, EP451216, EP682040, EP519596, EP460167, WO94044679 and WO9222653, for instance, the descriptions of which being incorporated by reference in their entireties.

A suitable a humanized CCR5-specific antibody is named ST6/34, which when expressed as an intrabody prevents surface expression of CCR5 as efficiently as the parental antibody (ST6). The humanized intrabody ST6/34 have the same effect as ST6 and in addition does not elicit an immune response, which is a potential complication in gene therapeutic delivery of a foreign protein.

In a second aspect, the invention covers also a recombinant antibody protein fused to an intracellular anchor means which is specific for a surface receptor necessary for pathogenic infection. The details described above are mutandis mutatis applicable and include the above mentioned preferred embodiments, i.e. a scFv-fusion protein comprising a scFv domain that immunoreacts with CCR5 or CXCR4 fused to an intracellular anchor means, and a scFv-fusion protein comprising at least a chain selected from VH and VL of ST6 or ST6/34.

In a third aspect, the invention covers also any recombinant antibody that immunoreacts with CCR5 or CXCR4, in particular antibodies providing CDRs or hypervariable loops from ST6 or ST6/34 antibodies, as shown in SEQ ID NO:1 (ST6 VL) and SEQ ID NO:2 (ST6 VH), SEQ ID NO:3 (ST6/34 VL) and SEQ ID NO:4(ST6/34 VH), and as defined in FIGS. 1A and 1B. Suitable antibodies are those comprising variable regions of ST6 and ST6/34 antibodies. In a preferred embodiment these antibodies are humanized as described above. They can be also contacted in vivo with a CCR5-bearing cell by administering the composition intravenously (i.v.) to a patient, and thereby presenting the antibody to available cells in the patient. Typically, the composition is injected i.v. into the patient in an amount sufficient to present a serum concentration to contact available cells with an effective amount of antibody.

In a fourth aspect, the invention covers also a polynucleotide molecule that encodes an antibody or a fusion protein of the present invention. This includes genes encoding such antibody or fusion protein, or vectors encoding such genes, including viral expression systems.

A vector suitably comprises nucleotide sequences which encode an antibody or fusion protein of the present invention operatively linked to expression control sequences necessary for the expression of the encoded protein. Preferably, a vector comprises expression control sequences, i.e., promoters, for expression in eucaryotic cells. Vectors containing both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). Examples of vectors include vectors derived from viruses, such as baculovirus, retroviruses, adenoviruses, adeno-associated viruses, and herpes simplex viruses; bacteriophages; cosmids; plasmid vectors; fungal vectors; synthetic vectors; and other recombination vehicles typically used in the art. These vectors have been described for expression in a variety of eukaryotic and prokaryotic hosts and may be used for protein expression.

In a preferred embodiment, the viral vector comprises a nucleic acid sequence coding for an antibody or a fusion protein according to the invention, operatively linked to an expression control sequence. Selection of appropriate control sequences is dependent on the target cell used and the choice is within the skill of one in the art. Examples of expression control sequences, also referred to as regulatory sequences, include promoters, enhancers, polyadenylation signals, RNA polymerase binding sequences, sequences conferring inducibility of transcription and other expression control elements, such as scaffold attachment regions (SARs).

The promoter may be either a prokaryotic or eukaryotic promoter. Additionally the promoter may be a tissue specific promoter, inducible promoter, synthetic promoter, or hybrid promoter. More than one promoter may be placed in the construct. Examples of promoters include but are not limited to the phage lamda (PL) promoter; SV40 early promoter; adenovirus promoters, such as adenovirus major late promoter (Ad MLP); herpes simplex virus (HSV) promoter; a cytomegalovirus (CMV) promoter; such as the human CMV immediate early promoter; a long terminal repeat (LTR) promoter, such as a MoMLV LTR; the U3 region promoter of the Moloney murine sarcoma virus; Granzyme A promoter; regulatory sequences of the metallothioein gene; CD34 promoter; CD8 promoter; thymidine kinase (TK) promoters; B19 parvovirus promoters; and rous sarcoma virus (RSV) promoter. Additionally promoter elements from yeast and other fungi may be used, such as Gal 4 promoter and the alcohol dehydrogenase (ADH) promoter. These promoters are available commercially from various sources such as Stratagene (La Jolla, Calif.). It is to be understood that the scope of the present invention is not to be limited to a specific promoter.

The vector may further comprise a polyadenylation signal that is positioned 3′ of the carboxy-terminal amino acid. Vectors containing both a promoter and a cloning site into which a polynucleotide can be operably linked are well known in the art. Such vectors are capable of transcribing RNA in vitro or in vivo, and are commercially available. Specific non-limiting examples include pSG, pSV2CAT, and pXt1 from Stratagene (La Jolla, Calif.) and pMSG, pSVL, pBPV and pSVK3 from Pharamacia. Other exemplary vectors include the pCMV mammalian expression vectors, such as pCMV6b and pCMV6c (Chiron Corporation, CA), pSFFV-Neo, and pBluescript-SK+. In order to optimize expression and/or in vitro transcription, it may be necessary to remove, add or alter 5′and/or3′ untranslated portions of polynucleotides to eliminate potentially extra inappropriate alternative translation initiation codons or other sequences that may interfere with or reduce expression, either at the level of transcription or translation. Alternatively consensus ribosome binding sites can be inserted immediately ‘5’ of the start codon to enhance expression.

Particularly preferred vectors are retroviral vectors and reference is made to Coffin et al., “Retroviruses”, (1997) Chapter 9 pp; 437-473 Cold Springs Harbor Laboratory Press. Retroviral vectors useful in the invention are produced recombinantly by procedures already taught in the art. WO94/29438, WO97/21824 and WO97/21825 describe the construction of retroviral packaging plasmids and packing cell lines. Common retroviral vectors are those derived from murine, avian or primate retroviruses. The most common retroviral vectors are those based on the Moloney murine leukemia virus (MoMLV) and mouse stem cell virus (MSCV). Vectors derived from MoMLV include, LMily, LINGFER, MINGFR, MND and MINT (Bender et al., J. Virol., 61:1639-1649, 1987; Miller et al., Biotechniques, 7: 998-990,1989; Robbins et al., J. Virol. 71:9466-9474,1997; and U.S. Pat. No. 5,707,865). Vectors derived from MSCV include MSCV-MiLy (Agarwal et al., J. of Virology 72:3720). Further non-limiting examples of vectors include those based on Gibbon ape leukemia virus (GALV), Moloney murine sacroma virus (MoMSV), myeloproliferative sarcoma virus (MPSV), murine embryonic stem cell virus (MESV), spleen focus forming virus (SFFV) and the lentiviruses, such as Human immunodeficiency virus (HIV-1 and HIV-2). New vector systems are continually being developed to take advantage of particular properties of parent retroviruses such as host range, usage of alternative cell surface receptors and the like (See C. Baum et al., Chapter 4 in Gene Therapy of Cancer Cells eds., Lattime and Gerson (1998)). The present invention is not limited to particular retroviral vectors, but may include any retroviral vector. Particularly preferred vectors include DNA from a murine virus corresponding to two long terminal repeats, and a packaging signal. In one embodiment the vector is a MoMLV or MSCV derived vector. In another preferred embodiment the vector is MND.

In producing retroviral vector constructs, the viral gag, pol and env sequence will generally be removed from the virus, creating room for insertion of foreign DNA sequences. Genes encoded by foreign DNA are usually expressed under the control a strong viral promoter in the long terminal repeat (LTR). While a LTR promoter is preferred, as mentioned above, numerous promoters are known.

Such a construct can be packaged into viral particles efficiently if the gag, pol and env functions are provided in trans by a packaging cell line. Therefore when the vector construct is introduced into the packaging cell, the gag-pol and env proteins produced by the cell, assemble with the vector RNA to produce infectious virions that are secreted into the culture medium. The virus thus produced can infect and integrate into the DNA of the target cell, but does not produce infectious viral particles since it is lacking essential packaging sequences. Most of the packaging cell lines currently in use have been transfected with separate plasmids, each containing one of the necessary coding sequences, so that multiple recombination events are necessary before a replication competent virus can be produced. Alternatively the packaging cell line harbors a provirus. (The DNA form of the reverse-transcribed RNA once its integrates into the genomic DNA of the infected cell). The provirus has been crippled so that although it may produce all the proteins required to assemble infectious viruses, its own RNA can not be packaged into virus. RNA produced from the recombinant virus is packaged instead. Therefore, the virus stock released from the packaging cells contains only recombinant virus. Non-limiting examples of retroviral packaging lines include PA12, PA317, PE501, PG13, ΨCRIP, RD114, GP7C-tTA-G10, ProPak-A (PPA-6), and PT67. Reference is made to Miller et al. (Mol. Cell Biol., 6:2895,1986; Biotechniques, 7:980, 1989), Danos et al. (PNAS, 85:6460,1988), Pear et al. (PNAS, 90:8392,1993), Rigg, et al. (Virology, 218, 1996); and Finer et al. (Blood, 83:43,1994). Retroviral vector DNA can be introduced into packaging cells either by stable or transient transfection to produce vector particles.

Additionally preferred vectors include adenoviral vectors (See Frey et al., Blood 91:2781 (1998) and WO95/27071) and adeno-associated viral vectors (AAV) (See Chatterjee et al., Current Topics in Microbiol. and Immunol., 218:61 (1996). Reference is also made to Shenk, Chapter 6, 161-78, Breakefield et al., Chapter 8: 201-235; Kroner-Lux et al., Chapter 9: 235-256 in Stem Cell Biology and Gene Therapy, eds. Quesenberry et al., John Wiley & Sons, 1998, and U.S. Pat. Nos. 5,693,531 and 5,691,176. The use of adenovirus derived vectors may be advantageous under certain situations because they are capable of infecting non-dividing cells, and unlike retroviral DNA, the adenoviral DNA is not integrated into the genome of the target cell. Further the capacity to carry foreign DNA is much larger in adenoviral vectors than retroviral vectors. The adeno-associated viral vectors are another useful delivery system. The DNA of these viruses may be integrated into non-dividing cells, and a number of polynucleotides have been successfully introduced into different cell types using adeno-associated viral vectors. The vectors are capable of transducing several cell types including hematopoietic cells and epithelial cells.

Vectors may also include hybrid vectors of AAV and adenoviruses as described in WO96/13598 and WO99/47691 (The Trustees of the University of Pennsylvania), WO98/21345 (General Hospital), U.S. Pat. No. 5,965,441 (General Hospital), or WO99/58700 (Ariad Gne Therap.), the teaching of which being incorporated into the present invention in their entirety.

According to the first aspect of the invention, the above vectors can be directly introduced in vivo by administering a therapeutical amount of the vector to the body of a patient, i.e. by i.v. inoculation and the like means, where the vector has the properties of transforming one or more cell type, depending upon the nature of the vector. For example, adenovirus vectors are known which target specific cell types and thereafter infect that cell type and introduce the vector genes into the cell for expression. Thereafter, the vector expresses the encoded antibody construct intracellularly, thereby presenting antibody to intracellular CCR5 and/or CXCR4 and immunocomplexing with them before it is transported to the cell surface.

In a fifth aspect, the invention provides also peptides comprising at least YTSE or YTSQ amino acid sequence for use in a vaccine or an immunogenic composition intended to control, prevent, diminish or treat HIV infections. The invention also covers such vaccines or immunogenic compositions. The peptides may be chemically synthesized with at least 8 contiguous amino acid residues in length, to about 40 or more. A peptide may contain several YTSE and/or YTSQ motifs, preferably repeated in tandem. A peptide of the invention may also be derivatized to a suitable carrier protein to form a conjugate, and/or be combined with suitable adjuvants or stabilizers in amount generally used in vaccines as described herein under. A vaccination strategy may comprise administration of one or more of peptides. In general, the initial administration of an immunogenic peptide vaccine consists of at least 1 mg. Follow-up or “booster” administrations of immunogenic peptide vaccines are usually provided to patient in successive, spaced administrations.

In one preferred embodiment, peptides may be covalently coupled to the diphtheria toxo1d (DT) carrier protein via the cysteinyl side chain using approximately 15-20 peptide molecules per molecule of diphtheria toxoid (DT). In general, derivatized peptide vaccine compositions are administered with a vehicle. The purpose of the vehicle is to emulsify the vaccine preparation. Numerous vehicles are known to those of skill in the art, and any vehicle which functions as an effective emulsifying agent finds utility in the methods of the invention. One preferred vehicle for administration comprises a mixture of mannide monooleate with squalane and/or squalene. To further increase the magnitude of the immune response resulting from administration of the vaccine, an inummological adjuvant is preferably included in the vaccine ormulation. Exemplary adjuvants known to those of skill in the art include water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs. Preferred adjuvants for inclusion in an vaccine composition for administration to a patient are norMDP analogs, such as N-acetyl-nor-muranyl-L-alanyl-D-isoglutamine, N-acetyl-muranyl-(6-0-stearoyl)-L-alanyl-D-isoglutamine, and N-Glycol-muranyl-L.alphaAbu-D-isoglutamine (Ciba-Geigy Ltd.). In most cases, the mass ratio of the adjuvant relative to the peptide conjugate is about 1:2 to 1:20. In a preferred embodiment, the mass ratio of the adjuvant relative to the peptide conjugate is about 1:10. It will be appreciated that the adjuvant component of the vaccine may be varied in order to optimize the immune response to the immunogenic epitopes therein.

Suitable pharmaceutically acceptable carriers for use in an immunogenic proteinaceous composition of the invention are well known to those of skill in the art. Such carriers include, for example, phosphate buffered saline, or any physiologically compatible medium, suitable for introducing the vaccine into a subject.

Numerous drug delivery mechanisms known to those of skill in the art may be employed to administer the immunogenic peptides and of the invention. Controlled release preparations may be achieved by the use of polymers to complex or absorb the peptides or antibodies in the methods of the present invention. Controlled delivery may accomplished using macromolecules such as, polyesters, polyamino acids, polyvinyl pyrrolidone, ethylenevinylacetate, methylcellulose,carboxymethylcellulose, or protamine sulfate, the concentration of which can alter the rate of release of the peptide vaccine.

In some cases, the peptides may be incorporated into polymeric particles composed of e.g., polyesters, polyamino acids, hydrogels, polylactic acid, or ethylene vinylacetate copolymers. Alternatively, the hCG peptide vaccine is entrapped in microcapsules, liposomes, albumin microspheres, microemulsions, nanoparticles, nanocapsules, or macroemulsions, using methods generally known to those of skill in the art.

In a sixth aspect, the invention provides also antiidiotypic antibodies mimicking CCR5 or CXCR4 epitopes raised from anti-CCR5 and anti-CXCR4 antibodies. Idiotypes are serologically defined entities since injection of an antibody (often referred to as Ambi) into a syngeneic, allogeneic, or xenogeneic recipient induces the production of anti-idiotypic antibodies (often referred to as Ab2). Based on the assumption that idiotype/anti-idiotype interactions exist, physiologically a receptor-based regulation of the immune system was postulated by Niels Jerne (Ann. Immunol., 125C, 373, 1974). His network theory views the immune system as a collection of 1 g molecules and receptors on T-lymphocytes, each capable of recognizing an antigenic determinant (epitope) through its combining site (paratope), and each capable of being recognized by other antibodies or cell-surface receptors of the system through the idiotopes that it displays.

Many studies have indeed demonstrated that idiotypic and anti-idiotypic receptors are present on the surface of both B- and T-lymphocytes as well as on secreted antibodies. When the binding between Ab1 and Ab2 is inhibited by the antigen to which Ab1 is directed, the idiotype is considered to be binding-site-related, since it involves a site on the antibody variable domain that is engaged in antigen recognition. Those idiotypes which conformationally mimic an antigenic epitope are called the internal image of that epitope. Since both an Ab2 and an antigen bind to the relevant Abe1, they may share a similar three-dimensional conformation which represents the internal image of the given antigen. Internal image anti-idiotypic antibodies in principle can be seen as substitute of the antigen from which they have been derived via the idiotypic network. Therefore these surrogate antigens may be used in active immunization protocols. For example they offer advantages if the original antigen is not sufficiently immunogenic to induce a significant immune response. Thus, appropriate internal image antiidiotypic antibodies that mimic a non-immunogenic carbohydrate antigen may be especially useful for certain vaccination approaches.

As exposed in the following examples, the teaching of WO9324647 (Sandoz) can be readily carried out to make antiidiotypic antibodies generated from antibodies ST6 or humanized antibodies thereof, such as ST6/34.

The invention generally described above will be more readily understood by reference to the following examples, which are hereby included merely for the purpose of illustration of certain embodiments of the present invention and are not intended to limit the invention in any way. These examples are preceded by a brief description of the figures.

### FIGURES

FIG. 1A illustrates amino acid sequence alignment of the rabbit ST6 VL and the human clones selected during the light chain humanization. Clones 13A, 10A and 8A are (human) lambda light chains and have V segments of the VL2 family. Clone 12A is a (human) kappa light chain and its V segment was determined to be of the kIII subgroup.

FIG. 1B illustrates alignment of the rabbit ST6-VH sequence and the humanized ST6/34-VH sequence. ST6/34 has the 13A light chain.

FIG. 2 displays the peptide motives selected from the phage displayed peptide library aligned with the N-terminal extracellular domain of CCR5 (aa 1-33). The amino acid-sequence of five overlapping peptides (P1-P5) spanning the N-terminus of CCR5 is shown.

### EXAMPLE 1

### CCR5 Intrabodies

1.Materials

PM1 cells were grown in RPMI 1640 containing 10% FBS (fetal bovine serum) and antibiotics. Transduced PM1 cells were usually maintained in the presence of puromycin (0.5 μg/ml) except during cell-cell fusion assays and infection assays. COS7 cells and PA317 (both American Type Culture Collection) and 293T cells (obtained from R. W. Doms) were maintained in DMEM (Dulbecco's modified Eagle's medium) containing 10% FBS and antibiotics. Tissue culture media and reagents were from GibcoBRL.

The following vaccinia recombinants were used: vCB-21R (Lac Z gene) (Alkhatib et al., J Virol 70, 5487-5494, 1996); vTF7-3 (T7 RNA polymerase) (Fuerst et al., PNAS, 83: 8122-8126,1986); vCB-28 (JR-FL env) (O'Brien et al., Nature, 348: 69-73, 1990); vCB-32 (SF162 env) (Cheng-Mayer et al., J Virol, 64: 4390-4398, 1990); vCB43 (Ba-L env) (Hwang et al., Science 253: 71-74, 1991; Broder et al., Cell, 85: 1149-1158, 1996); vBD3 (89.6 env) (Dorank et al., Cell, 85:1149-1158, 1985); vCB 74 (SIV mac 239 env) (Edinger et al., PNAS, 94: 4005-4010, 1997). Infection and further treatment of the effector cells was done as described (Rucker et al., Methods Enzymol, 288: 118-133, 1997). The reporter R5 HIV-1 virus construct, NFN-SX-r-HSAS was obtained from B. D. Jamieson and J. A. Zach, but other reporter viruses could have been used.

Plasmids encoding human CCR5 and CXCR4 (Deng et al., Nature, 381: 661-666, 1996), and rhesus CCR5 and CD4 (Chen et al., Virology, 246:113-124, 1998) were obtained from the NIH AIDS Research and Reference Reagent Program, but are available from other sources. Plasmid encoding human CD4 was obtained from B. J. Doranz, but is available from other sources. Reporter plasmid containing the luciferase gene under the control of the T7 polymerase was purchased from Promega and plasmid pcDN0A3.1/Zeo was purchased from Invitrogen.

Antibodies specific to human CCR5, CXCR4, CD4 and RANTES were purchased from PharMingen. FITC or PE conjugated secondary antibodies were purchased from JacksonImmunoResearch Laboratories except for the anti rat-FITC conjugate which was obtained from PharMingen. A high affinity HA-tag specific monoclonal rat antibody was purchased from Roche Molecular Biochemicals. CCR5-specific antibody 5C7 (Wu et al., J Exp Med., 185: 1681-1691, 1997) was obtained from the NIH AIDS Research and Reference Reagent Program. CCR5-specific antibody ST6 was obtained as described in example 2.

2.Methods

2.1 Conversion of a CCR5 Specific Fab-clone Into a Single Chain Antibody Fragment (scFv)

Specific oligonucleotide primers were used to amplify V_{H} and V_{L} gene segments from purified phagemid DNA isolated from ST6 a Fab fragment specific for the N-terminal extra-cellular domain of CCR5. This Fab was isolated from an immunized rabbit using the phage display approach (Rader et al., 3rd Curr Opin Biotechnol, 8: 503-508, 1997). The following primers were used: V_{L}: RSCVK1 5′-GGGCCCAGGCGGCCGAGCTCGTGMTGACCCAGACTCCA-3′, RKB9J0-B 5′-GGAAGATCTAGAGGAACCACCTAGGATCTCCAGCTCGGTCCC-3′; V_{H}: RSCVH3 5′-GGTGGTTCCTCTAGATCTTCCCAGTCGYTGGAGGAGTCCGGG-3′, HSCG1234-B 5′CCTGGCCGGCCTGGCCACTAGTGACCGATGGGCCCTT GGTGGARGC-3′. The purified PCR products were assembled by another PCR reaction using the following primers: RSC-F 5′-GAGGAGGAGGAGGAGGAGGCGG GGCCCAGGCGGCCGAGCTC-3′, RSC-B 5′GAGGAGGAGGAGGAGGAGC CTGGCCGGCCTGGCCACTAGTG-3′.

The resulting overlap-PCR product encodes a scFv were the N-terminal V_{L} region is linked with the V_{H} region through a seven amino acid peptide linker (GGSSRSS). The DNA fragment was gel purified, digested with the restriction endonuclease Sfi-I, and cloned into the appropriately cut phagemid vector pComb3X, a variant of pComb3H (Rader et al., supra). Binding activity of the expressed scFv was confirmed and the gene encoding the scFv was transferred to pcDNA3.1/Zeo and pBabe Puro vectors.

2.2 Generation of pcDNA3.1/Zeo and pBabe Puro Intrabody and Intrakine Constructs.

Both pcDNA3.1/Zeo and pBabe Puro (Morgenstern et al., Nucleic Acids Res, 18: 3587-3596, 1990) were modified by introducing two Sfi-I sites into their multiple cloning sites. A human kappa leader sequence was cloned into the vectors upstream of the 5′ Sfi-I sites. Downstream of the 3′ Sfi-I site, a sequence encoding the HA-tag sequence (YPYDVPDYA) (Wilson et al., Cell, 37:767-778, 1984) and an ER retention signal (KDEL) followed by a stop codon was introduced. The ST6 scFv, as well as control scFv encoding DNA fragments were cloned into the appropriately digested vector DNAs. The modified pcDNA3.1/Zeo plasmid encoding ST6 was designated pIB6.

For control purposes an intrakine construct for intracellular expression of the CCR5 binding chemokine RANTES (Yang et al., 1997 supra) was also made: Human RANTES cDNA was PCR amplified using the primers :RANF 5′-GAGGAGGAGGAGGAGGCTAGCATGAAGGTCTCCGCGGCAC-3′ and, RAN-B-SfiI 5′-GGAACGTCGTACGGGTACTGGCCGGCCTGGCCGCTCATCTCCAAAG AGTTGATGT ACTCCCG-3′.

The PCR product was digested using NheI and SfiI and gel-purified. The RANTES encoding DNA insert was cloned into the modified and appropriately cut pcDNA 3.1/Zeo DNA. The resulting plasmid encoding the RANTES intrakine without the kappa leader sequence was designated pRAN. The sequence of the intrakine insert was confirmed by DNA sequence analysis.

2.3 Cotransfection of 293T Cells Using Chemokine Receptor Encoding Plasmid and Intrabody or Intrakine Encoding Plasmid.

293T cells were transfected using LipofectAmine (GibcoBRL) according to the manufacturer's protocol with plasmids containing coreceptor genes. At the same time, cells were cotransfected with 2-fold molar excess of plasmid encoding CCR5 specific intrabody (pIB6) or intrakine (pRAN) or with control plasmid—pcDNA 3.1/Zeo containing no insert.

2.4 Flow Cytometric Analysis of Cotransfected 293T Cells and Transduced PM1 Cells.

For surface staining, cells were incubated with primary antibodies for 30 minutes, washed and stained with appropriate FITC or PE conjugates. For intracellular staining, cells were permeabilized with phosphate buffered saline (PBS) containing 4% paraformaldehyde (Sigma) and 0.1% saponin (Sigma) for 10 minutes and washed. Cells were then incubated with primary antibodies for 30 minutes, washed and stained with appropriate FITC or PE conjugates. Throughout the staining, the washing and staining buffers contained saponin (0.1%). After staining and washing, the cells were resuspended in PBS without saponin. Cells were analyzed on Becton Dickinson Flow cytometers (FACScan, FACSort or FACSCalibur) using Cell Quest software.

2.5 Reporter Gene Fusion Assay.

A modified reporter gene assay was used to quantify cell-cell fusion events (Rucker et al., 1997 supra; Nussbaum et al., J Virol, 68: 5411-5422, 1994). Briefly, T7 RNA polymerase and HIV-1 or simian immunodeficiency virus (SIV) env were introduced into 293T effector cells using vaccinia virus recombinants. COS7 cells, used as target cells, were transfected with a plasmid encoding luciferase under the control of a T7 promoter and plasmids containing human or rhesus CCR5 genes, and human or rhesus CD4 genes using LipofectAmine. Target cells were cotransfected with plasmid encoding ST6 intrabody (pIB6) or plasmid encoding RANTES intrakine (pRAN) or control plasmid pcDNA3.1/Zeo containing no insert. To assure cotransfection, the plasmids encoding intrabody or control plasmids, were introduced in 2-fold molar excess over the plasmids encoding the coreceptors. To assess background luciferase activity, a set of target cells was transfected with luciferase- and CD4-encoding plasmids but not CCR5-encoding plasmid. Duplicate transfection mixes were setup for each kind of target cell and each transfection mix was distributed to two wells. After overnight incubation, effector cells were added to wells containing target cells and cocultured for 8-10 hours in the presence of rifampicin (100 μg/ml, Sigma) and araC (cytosine β-D-arabinofuranose, 10 μM, Sigma). Cells were then lysed and assayed for luciferase activity. When using transduced and untransduced PM1 cells as target cells in a reporter gene fusion assay, the cells were infected with a vaccinia recombinant encoding the T7 RNA polymerase. 293T cells that were used as effector cells were transfected with luciferase reporter plasmid and infected with vaccinia virus recombinants encoding HIV-1 env. To assess background luciferase activity, a set of target cells was infected with a control vaccinia recombinant containing the lacZ gene.

2.6 Retroviral Gene Transfer: Generation of Transduced PM1 Cells

The amphotropic packaging cell line PA317 (Miller et al., Mol Cell Biol, 6: 2895-2902, 1986) was transfected with pBabe Puro plasmids encoding the ST6 scFv insert. For control purposes pBabe Puro plasmids encoding scFv specific to Glutathione S-transferase (GST) or human integrin ανβ3 (RAI3) were also used. Producer lines were selected by adding 2 μg/ml Puromycin to the cultures. These stable lines were used to generate virus-containing medium for 2 rounds of infection of PM1 cells in the presence of 8 μg/ml polybrene (Sigma). Two days after the last infection, transduced PM1 cells were selected in puromycin (0.5 μg/ml). After 14 days of selection, analysis of cells for CCR5 expression and infectability was started. The untransduced parental PM1 cell line was named PM1-P and the PM1 cells transduced to express ST6 intrabody were named PM1-6. The PM1 cell lines transduced to express the control intrabodies RAI3 and a GST-specific intrabody, were named PM1-RAI3 and PM1-GST, respectively. For some experiments transduced intrabody expressing PM1 clones obtained from limiting dilution cultures were used. PM1-6-A2 and PM1-6-G were cloned from the PM1-6 cell line and PM1-RAI3-5 was cloned from the PM1-RAI3 cell line.

2.7 HIV-1 Infection of the PM1 Cells.

Transduced and untranduced PM1 cells were infected at an multiplicity of infection of 0.01 for 5 hours at 37° C., washed, and then cultured for up to 16 days. To monitor infection, aliquots were taken from the cultures at the indicated time points and p24 levels were determined in a HIV-1 ELISA (NEN™ Life Sciences).

2.8 Cocultivation of PM1 Cells With Infected Parental PM1 Cells.

Parental PM1 were infected with the NFN-SX-r-HSAS reporter virus. In this virus the HIV-1 vpr is replaced with murine HSA (heat stable antigen, CD24), allowing infected cells to be monitored by flow cytometry. Here, a virus was constructed by replacing the env of NFN-SX-r-HSAS (Jamieson et al., J Virol, 72 6520-6526, 1998) with the env sequence of the CCR5-using JR-FL. When about 5% of the cells were infected, three cocultures were initiated: A 2-fold excess of cells from the infected PM1-P culture were mixed with PM1-P cells and with the transduced PM1 cell clones PM1-RAI3-5 and PM1-6-G. Cocultures were monitored for intrabody and HSA expression by flow cytometry. To reduce false positives, doublets or larger aggregates of PM1 cells were excluded from analysis.

3. Results

3.1 Transfection of a ST6-encoding Plasmid (pIB6) Blocks Surface Expression of CCR5.

The ability of ST6 intrabody to block rhesus CCR5 expression was studied to assess whether it could be used in non-human lentivirus models. CCR5 is the primary coreceptor for SIV and the N-terminal extracellular domain sequence of rhesus CCR5 has only two amino-acid substitutions as compared to the human CCR5 sequence (Villinger et al., 1999 supra). An antibody fragment, ST6, which binds the N-terminal extracellular domain of CCR5 was originally derived from a Fab phage display library. ST6 was converted into a scFv where the V_{L} and the V_{H} fragments were covalently linked with a peptide linker consisting of seven amino acids. Upon expression, use of this short peptide linker results in dimeric scFv proteins (Zhu et al., Biotechnology,14: 192-196, 1996). To retain the antibody fragment in the ER, an ER retention peptide (KDEL) was appended to the C-terminus of the protein (Munro et al., Cell, 4.8: 899-907, 1987). We anticipated that ST6 scFv dimers expressed within cells as intrabodies possessing two functional CCR5 binding sites and two ER retention signals would efficiently trap CCR5 proteins en route to the cell surface via their natural ER trafficking pathway. As a control protein, the C-C-chemokine RANTES was cloned into the same expression vector as a fusion with the ER retention sequence as previously described (Yang et al., 1997 supra). RANTES expressed in this manner has been shown to be retained predominately in the ER and has been termed an intrakine.

The effect of intrabody and intrakine coexpression on the surface expression of human and rhesus CCR5 was examined by flow cytometry. Upon transfection with a CCR5-encoding plasmid, 293T cells expressed high levels of CCR5. To study the effect of expression of the intrabody and intrakine, cotransfections were performed using a 2-fold molar excess of control plasmid (pcDNA3.1/Zeo) or plasmid encoding ST6 (pIB6) or RANTES (pRAN). Upon cotransfection with pIB6-DNA, no surface expression of human CCR5 was detected by flow cytometry, whereas cotransfection with pRAN resulted in only a slight reduction of CCR5 expression. Intracellular staining using an antibody specific to the HA-tag encoded in the expression plasmids upstream of the KDEL sequence showed that the expression levels for the intrabody and the intrakine were comparable. Intracellular expression of RANTES in cells cotransfected with pRAN was also confirmed by incubating permeabilized cells with a RANTES specific antibody. The ability of ST6 intrabody to block rhesus CCR5 expression was studied to assess whether it could be used in non-human lentivirus models. CCR5 is the primary coreceptor for SIV and the N-terminal extracellular domain sequence of rhesus CCR5 has only two amino-acid substitutions as compared to the human CCR5 sequence (Villinger et al., 1999 supra). Transfection studies of 293T cells were performed as described above with a plasmid encoding rhesus CCR5 replacing that used for human CCR5 expression. Again cotransfection with ST6 scFv encoding plasmid pIB6 completely blocked transient rhesus CCR5 expression, whereas pRAN had little effect. Note that rhesus RANTES is identical in sequence to the human chemokine (Villinger et al., 1999 supra). Cotransfection studies using plasmids encoding human CXCR4 with pIB6 or pRAN demonstrated that neither pIB6 nor pRAN affected transient CXCR4 expression. Intracellular HA and RANTES staining confirmed that both, the intrabody and the intrakine were expressed at similar levels in these experiments. Cotransfection studies of the CCR5-expressing plasmid with plasmid encoding an irrelevant intrabody had no effect on CCR5 surface expression

3.2 ST6 Encoding Plasmid (pIB6) Prevents CCR5 Dependent Cell-cell Fusion.

The effect of ST6 intrabody expression on CCR5 dependent cell-cell fusion was investigated using a reporter gene assay. Plasmids encoding luciferase under the control of the T7 promoter (reporter plasmid) and human or rhesus CD4 and CCR5, were introduced into COS7 cells to generate two target cell populations. These cells were also cotransfected with ST6 plasmid pIB6 or with control plasmid. In some experiments, target cells were cotransfected with RANTES intrakine encoding plasmid pRAN. Effector cells of five types were prepared that expressed T7 RNA polymerase and env derived from either one of three different R5 HIV-1 variants, the R5X4 HIV-1 strain 89.6 or a SIV strain. Effector cells were then cocultured with the target cells. In this assay, measurement of luciferase activity allows cell-cell fusion activity to be quantified (Rucker et al., 1997 supra). We demonstrated that cotransfection with pIB6 reduced CCR5-dependent cell fusion to background levels. Fusion assays were repeated at least twice with similar outcomes. Cotransfection with plasmid encoding the RANTES intrakine, pRAN produced only a slight reduction of cell fusion activity. Cotransfection of pIB6-DNA with CXCR4 encoding plasmid did not affect CXCR4 dependent cell fusion.

3.3 Generation and Characterization of a ST6 scFv Expressing PM1 Cell line.

Recombinant retroviruses encoding intrabody ST6 or control intrabodies were used to transduce the CCR5⁺/CD4⁺-human lymphocyte cell line PM1. Transduced PM1 cell lines were established through puromycin selection. Parental cells, transduced cell lines and clones were analyzed for CCR5 expression and intrabody expression by flow cytometry. The untransduced parental cell line and PM1 cells transduced with retrovirus directing the expression of a control intrabody that does not bind CCR5, expressed CCR5 on their cell surface as was reported previously (Wu et al., 1997 supra). In contrast, no CCR5 surface-expression could be detected with PM1-6, the PM1 cell line tranduced with ST6 intrabody encoding retrovirus. Intrabody could be detected by staining permeabilized transduced PM1 cells with an anti-HA antibody. The PM1-6 line showed homogeneous and stable expression of intrabody after puromycin selection, whereas only about 30% of PM1 cells transduced with control intrabody (PM1-RAI3) encoding retrovirus stained positively for intrabody expression. Therefore, limiting dilution cloned transduced PM1 lines and a control intrabody-expressing clone (PM1-RAI3-5) was used in some experiments. For comparative studies two clones derived from the PM-6 line, PM1-6-G and PM1-6-A2 were also isolated. No single-chain antibody was detected on the surface of unpermeabilized transduced PM1 cells using the same primary and secondary antibodies used for intracellular detection. PM1-6 culture supernatants were also examined for the presence of CCR5 specific scFv by ELISA using purified ST6 scFv as a reference. In this assay (sensitivity ˜2 ng scFv/ml) no scFv was detected.

3.4 PM1 Cells Expressing CCR5 Specific Intrabody ST6 are Protected From env Induced CCR5-dependent Cell Fusion.

Parental PM1 cells (PM1-P) and transduced PM1 clones were analyzed for their interaction with HIV-1 env expressing cells in a cell-cell fusion reporter assay. In this assay, transduced or untransduced PM1 target cells were infected with recombinant vaccinia virus expressing T7 RNA polymerase. These cells were subsequently cocultured with effector 293T cells that had been transfected with a luciferase reporter plasmid and infected with recombinant vaccinia virus that directs the expression of env derived from the R5 HIV-1 strain JR-FL. The background level of luciferase activity was established using target cells infected with recombinant vaccinia virus expressing β-galactosidase instead of T7 RNA polymerase (PM1-P-lacZ). Cell-cell fusion that resulted from the interaction of env expressing effector cells with the CCR5⁺/CD4⁺-untransduced line, PM1-P, and the transduced control PM1 cells (PM1-RAI3-5), was quantified by luminometry. We showed that no cell-cell fusion above background was detected after incubation of PM1-6-G cells with effector cells. In contrast, incubation of PM1-6-G cells with effector cells expressing env protein derived from 89.6, a HIV-1 strain that can also use CXCR4 as a coreceptor, led to cell fusion.

This demonstrates that PM1 cells expressing CCR5 specific intrabody ST6 are protected from R5 HIV-1 virus infection.

To verify the results of the fusion experiments, we challenged the intrabody expressing PM1 cell lines with R5 HIV-1 isolates. We showed that the parental PM1 cell line, PM1-P, was readily susceptible to infection with the R5 strains SF162 and JR-CSF as demonstrated by increasing p24 levels in the tissue culture supernatant (Experiment 1). In contrast, p24 levels in cultures of ST6 intrabody expressing PM1 cells (PM1-6-G) remained below the detection limit of 20 pg/ml p24 over the 10 day course of the experiment. As a control for non-specific intrabody effects, a PM1 line expressing an anti-GST intrabody was included in the second experiment. This control line was as readily infected with SF162 as were the parental PM1 cells and the p24 protein production that resulted from this infection closely tracked that observed with the parental PM1 line. The intrabody expressing line PM-6 as well as two clones derived from this line, PM1-6-G and PM1-6-A2, did not show any detectable p24 at any time during the 16-day time course of this experiment.

Independent studies of transduced PM1 and Jurkat lines expressing RANTES or SDF-1α intrakines, respectively, have shown that they are susceptible to low level infection and viral replication (Yang et al., 1997 supra; Chen et al., 1997 supra). To challenge the intrabody expressing PM1 cell lines with high amounts of virus under more stringent conditions, we cocultivated infected parental PM1 cells with the ST6 expressing PM1 cell clone PM1-6-G. In order to monitor infection of cells, a reporter virus, NFN-SX-r-HSAS was used. The reporter virus construct encodes JR-FL env and carries murine HSA as a vpr replacement. Cells infected with this virus can be detected by surface staining for HSA. In this experiment a 2-fold excess of PM1-P cells infected at the level of 5% were added to uninfected PM1 cells and transduced PM1 cell clones. Cells were stained for intrabody expression and for reporter virus infection using HA and HSA specific antibodies, respectively. In the coculture of parental PM1 cells with infected cells, the number of infected cells increased from 55% on day 3 to over 92% on day 7. In contrast, when infected PM1 cells were added to the (HA⁺) ST6 expressing cell line, PM1-6-G, virtually all HA⁺ cells remained HSA⁻ through the 20-day time course of the experiment. In this culture, the number of HSA⁺ cells on day 3 is higher (24%) than on day 7 (11%). This is likely to be due to a depletion of infectable PM1-P cells in the culture since on day 7, 85% of the HSA⁻ cells (75% of the total cell population) stain positive for HA. The very low number of HA⁺/HSA⁺ positive cells (0.2%) are probably false positives, since a similar number of cells stained HA⁺ in the coculture of infected cells with intrabody-negative (HA⁻) PM1-P cells. Furthermore, selection against untransduced PM1-P cells in the coculture using puromycin led to the loss of all HSA⁺ cells. Thus, even when exposed to R5 HIV-1 virus and infected cells for a prolonged period of time, PM1-6-G cells were completely resistant to infection. A transduced clone PM1-RAI3-5 expressing an irrelevant intrabody that was included as a control was readily infected by the reporter virus. PM1-6-G cells were susceptible to infection by an otherwise identical reporter virus expressing an X4 env.

Cocultivation of PM1-P cells with transduced PM1 cell lines demonstrated that both cell types had similar growth rates, since the proportion of intrabody expressing PM1 cells was found to be stable when analyzed by intracellular staining using a HA-tag specific antibody. This result showed that intracellular antibody expression has no obvious negative effects on cell viability or proliferation.

### EXAMPLE 2

### Humanization of CCR5 Intrabodies

1.Materials

293T cells were maintained in DMEM (Dulbecco's modified Eagle's medium) containing 10% FBS and antibiotics. Tissue culture media and reagents were from GibcoBRL.

Plasmids encoding human CCR5 and CXCR4 [Deng et al., 1996 supra], were obtained from the NIH AIDS Research and Reference Reagent Program. Plasmid encoding human CD4 was obtained from B. J. Doranz and plasmid pcDNA3.1/Zeo was purchased from Invitrogen.

Unconjugated and HRP-conjugated high affinity HA-tag specific monoclonal rat antibody was purchased from Roche Molecular Biochemicals. HRP-conjugated Donkey anti human IgG antibodies were purchased from JacksonImmunoResearch Laboratories. HRP-conjugated anti M13 phage antibody was from Amersham Pharmacia Biotech. PE-conjugated antibodies specific to human CCR5, CXCR4, and CD4 and an anti rat-IgG-FITC-conjugate were purchased from PharMingen. All other FITC- or PE-conjugated secondary antibodies were purchased from JacksonImmunoResearch Laboratories.

TABLE 1
V-specific oligonucleotide primers used for the
ST6 humanization: R = A or G; Y = C or T;
M = A or C; K = G or T; S = Gor C; W = A or T

Vκ sense primers (FR1-specific):

| | |
|---|---|
| HSCK1-F | 5′GGGCCCAGGCGGCCGAGCTCCAGATGACCCAGTCTC |
| | C 3′ |
| HSCK24-F | 5′GGGCCCAGGCGGCCGAGCTCGTGATGACYCAGTCTC |
| | C 3′ |
| HSCK3-F | 5′GGGCCCAGGCGGCCGAGCTCGTGWTGACRCAGTCTC |
| | C 3′ |
| HSCK5-F | 5′GGGCCCAGGCGGCCGAGCTCACACTCACGCAGTCTC |
| | C 3′ |

Vλ sense primers (FR1-specific):

| | |
|---|---|
| HSCLam1a | 5′GGGCCCAGGCGGCCGAGCTCGTGBTGACGCAGCCGCCCT |
| | C 3′ |
| HSCLam1b | 5′GGGCCCAGGCGGCCGAGCTCGTGCTGACTCAGCCACCCT |
| | C 3′ |
| HSCLam2 | 5′GGGCCCAGGCGGCCGAGCTCGCCCTGACTCAGCCTCCCT |
| | CCGT 3′ |
| HSCLam3 | 5′GGGCCCAGGCGGCCGAGCTCGAGCTGACTCAGCCACCCT |
| | CAGTGTC 3′ |
| HSCLam4 | 5′GGGCCCAGGCGGCCGAGCTCGTGCTGACTCAATCGCCCT |
| | C 3′ |
| HSCLam6 | 5′GGGCCCAGGCGGCCGAGCTCATGCTGACTCAGCCCCACT |
| | C 3′ |
| HSCLam70 | 5′GGGCCCAGGCGGCCGAGCTCGGGCAGACTCAGCAGCTCT |
| | C 3′ |
| HSCLam78 | 5′GGGCCCAGGCGGCCGAGCTCGTGGTGACYCAGGAGCCMT |
| | C 3′ |
| HSCLam9 | 5′GGGCCCAGGCGGCCGAGCTCGTGCTGACTCAGCCACCTT |
| | C 3′ |

Vκ reverse primers (specific for the 3′ end
of FR3):

| | |
|---|---|
| BKFR3UN | 5′CAGTAATACACTGCAAAATCTTC 3′ |
| BK2FR3UN | 5′CAGTAATAAACCCCAACATCCTC 3′ |
| BK3FR3UN | 5′CAGTAATAAGTTGCGAAATCATC 3′ |

Vλ reverse primers (specific for the 3′ end
of FR3):

| | |
|---|---|
| BLFR3 | 5′GCAGTAATAATCAGCCTCRTC 3′ |
| BLFR3New: | 5′CAGTAATAATCAGCCTCRTC 3′ |

Vκ sense (encoding the ST6-LCDR3 and flanked by
human FR3 and FR4 regions):

| | |
|---|---|
| KFR3 | 5′GAAGATTTTGCAGTGTATTACTGCGCAGGCGCTTATAGT |
| | GGTGATAGTGTTTTTGGCCAGGGGACCAAGCTG 3′ |
| K2FR3 | 5′GAGGATGTTGGGGTTTATTACTGCGCAGGCGCTTATAGT |
| | GGTGATAGTGTTTTTGGCCAGGGGACCAAGCTG 3′ |
| K3FR3 | 5′GATGATTTCGCAACTTATTACTGCGCAGGCGCTTATAGT |
| | GGTGATAGTGTTTTTGGCCAGGGGACCAAGCTG 3′ |

V_{H} sense (encoding the ST6-LCDR3 and flanked by
human FR3 and FR4 regions):

| | |
|---|---|
| LFR3 | 5′GAYGAGGCTGATTATTACTGCGCAGGCGCTTATAGTGGT |
| | GATAGTGTTTTCGGCGGAGGGACCAAGCTG 3′ |

V_{H} sense primers (FR1-specific):

| | |
|---|---|
| HSCVH1-F | 5′GGTGGTTCCTCTAGATCTTCCCAGGTGCAGCTGGTGCAG |
| | TCTGG 3′ |
| HSCVH2-F | 5′GGTGGTTCCTCTAGATCTTCCCAGATCACCTTGAAGGAG |
| | TCTGG 3′ |
| HSCVH35-F 5′GGTGGTTCCTCTAGATCTTCCGAGGTGCAGCTGGTGSAG | |
| | TCTGG 3′ |
| HSCVH3a-F | 5′GGTGGTTCCTCTAGATCTTCCGAGGTGCAGCTGKRGGAG |
| | TCTG 3′ |
| HSCVH4a-F | 5′GGTGGTTCCTCTAGATCTTCCCAGGTGCAGCTACAGCAG |
| | TGGGG 3′ |
| HSCVH4-F | 5′GGTGGTTCCTCTAGATCTTCCCAGGTGCAGCTGCAGGAG |
| | TCGGG3′ |

V_{H} sense primers (encoding the ST6-HCDR3 and
flanked by human FR3 and FR4 regions):

| | |
|---|---|
| HFR3 | 5′GACACGGCCGTGTATTACTGTGCGCGTGGGAATCCTGGT |
| | TGGGGTAGTGTCGTCTGGGGCCAGGGAACCCTG 3′ |

V_{H} reverse primer (specific for the 3′ end
of FR3):

| | |
|---|---|
| BFR3UN | 5′CGCACAGTAATACACGGCCGTGTC 3′ |

V_{H} reverse primer (specific for the 3′ end of FR2;
has a sequence tail specific for ST6-HCDR2):

| | |
|---|---|
| HC4-S | 5′GCACTGCTCGCGTATGCAGTGTTACCACTAGCGTAAATG |
| | ATTCCAATCCACTCCAGCCCCTTCCC 3′ |

V_{H} sense primers (specific for the ST6-HCDR2):

| | |
|---|---|
| HC5-S | 5′CACTGCATACGCGAGCAGTGCAAAAGG 3′ |

2. Methods

2.1 Generation of Fusion Proteins Representing the N-terminal Domains of Chemokine Receptors:

Overlapping oligonucleotides were used to generate a synthetic gene encoding the N-terminal extracellular domain of human CCR5 (amino acids 1-33) by PCR. Sfi I sites were introduced into 3′ and 5′ ends of the PCR products via the 3′ and the 5′ PCR primers. The bacterial expression vectors pMal-P2 (New England Biolabs) and pGEX-4T-1 (Amersham Pharmacia Biotech) were modified by introducing two assymetrical SfiI sites, respectively. The PCR product was SfiI cut and cloned into the SfiI cut vectors to generate expression plasmids encoding fusion proteins that have the CCR5-N-terminus fused their c-terminal end. The resulting fusion proteins named CCR5-N-GST and CCR5-N-MBP were expressed and purified following the manufacturers' protocols. Fusion proteins containing the N-terminal extracellular domain (amino acid 1-33) of CXCR4/Fusin were generated in a similar manner and called CXCR4-N-GST and CXCR4-N-MBP. These proteins were used as control protein in some experiments. Additional fusion-proteins with smaller peptides fused to the c-terminus of the MBP were generated as described above and named according the peptide sequence that was fused to the MBP-c-terminus. The following fusion proteins were made: MBP-YYTSEPG; MBP-YTSEG; MBP-YTSG; MBP-YTSDG; MBP-YTSQG. The integrity and purity of the fusion proteins was confirmed by SDS-PAGE analysis and Comassie-staining. The concentration was measured using a Bradford assay.

2.2 Rabbit Immunization:

A rabbit (New Zealand White) was treated with 4 subcutaneous injections containing 50 μg of purified CCR5-N-GST protein in a 1-ml emulsion of Ribi adjuvant in PBS (Ribi Immunochem Research, Hamilton, Mont.). The injections were administered in 2-3 week intervals. Sera from the immune animal were analyzed for binding to CCR5-N-GST and CCR5-N-MBP by ELISA and by flow cytometry. Five days after the final boost, spleen and bone marrow from one leg were harvested and used for total RNA preparation.

2.3 RNA Isolation, cDNA Synthesis, PCR Amplification and SfiI-cloning:

Human bone marrow aspirated from six healthy volunteers was purchased from Poietic Technologies (Germantown, Md.). Total RNA was prepared from human and rabbit tissue using TRI REAGENT from Molecular Research Center (Cincinnati, Ohio) according the manufacturers' protocol and was further purified by lithium chloride precipitation (Sambrook et al., 1989 supra). First-strand cDNA was synthesized using the SUPERSCRIPT Preamplification System for First Strand cDNA Synthesis kit with oligo(dT) priming (Life Technologies) according the manufacturers' protocol.

All PCR reactions were carried out in a volume of 100 μl and contained 60 pMoles of sense and 60 pMoles of reverse primers. The following temperature cycle was used: Denaturation at 94° C. for 30 seconds, anealing at 56° C. for 15 seconds and extension at 72° C. for 90 seconds. The extension time was increased to 120 or 180 seconds if the expected product size exceeded 600 bp or 1200 bp, respectively. AmpliTag polymerase was used in all PCR reactions except for PCR fragments larger than 1200 bp, were the Expand™ (High Fidelity) thermostable polymerase mix was used (both from Roche Molecular Biochemicals). For the amplification of expressed V-genes from first-strand-cDNA (1.5 μl), 30 cycles were performed. When DNA fragments were amplified form plasmids, 20 ng of template DNA was used and 20 cycles were performed. Overlap extension of two PCR products was done for 10 to 15 cycles using 50 ng of each fragment. For the construction of antibody libraries at least ten overlap-extension PCR reactions of one kind were performed. All PCR products were gel-purified for downstream procedures.

Final PCR-fragments encoding a library of antibody fragments were SfiI cut, purified and cloned into SfiI cut and purified phagemid DNAs pComb3H (Rader et al., Curr Opin Biotechnol, 8: 503-508, 1997) or pComb3X. pComb3X is a variation of pComb3H.

Antibody containing inserts that were used as PCR-templates for humanization-library construction, were subcloned into pho-plasmid vector DNA using SfiI sites. This was done to prevent phagemid contaminations in the constructed libraries.

2.4 Construction of a Chimeric Rabbit Antibody Library:

The sequences of the oligonucleotide primers used for the construction of the chimeric rabbit antibody library are given in Table I above. Rabbit V_{H} sequences were PCR amplified from first strand cDNA using four VH sense primers specific for the rabbit 5′ end of the rabbit V_{H} region and one 3′ primer specific for the JH region. The rabbit V_{H} sense primers have a 5′-sequence tail that is specific for the 3′ end of the pelB leader sequence. Vκ sequences were PCR amplified using three sense primers specific of the 5′ end of rabbit Vic sequences and three primers specific for the Jκ region. Vλ sequences were amplified using one Vλ sense and one Jλ reverse primer (Table 1 above). Separate PCR reactions were carried out with each primer combination. The reverse primers that were used to generate the rabbit V_{H}- and V_{L}-PCR products had sequence tails that were specific for the 5′ end of the human C_{H} and Cκ regions, respectively. A fragment containing the human Cκ region and the pelB sequence were amplified using a phagemid-vector containing a human Fab insert (pComb3XTT) as a template using the primer combination HKC-F and lead-B. The product of the pel B sequence that is located downstream of the Cκ region serves as a leader sequence of the heavy chain fragment in the Fab-phagemid vector. The human CH1 region was also amplified from the same pagemid DNA using the sense primer HIgGCH1-F and the reverse primer dpseq. The rabbit VL fragment and the human CL-pelB fragment were fused through an overlap extension PCR using the sense extension primer RSC-F, and the reverse primer lead-B. In a similar manner, the rabbit V_{H} fragments were fused to the PCR products encoding the human C_{H} region using the primer combination lead-VH (sense) and dpseq (reverse). In a final overlap extension PCR step, the products encoding the chimeric light chains and the pelB peptide were fused with the PCR products encoding the chimeric heavy chains using the extension primers RSC-F and dp-EX. The final 1500 bp PCR-product was cloned into the phagemid vector pComb3H using 2 asymmetric SfiI sites.

2.5 Light Chain Humanization:

The sequences of the VL-specific oligonucleotides primers used for the humanization of the ST6-light chain are given in Table 1 above. Human V κ genes were amplified using first strand cDNAs from human bone marrow obtained from six human volunteers using sense primers specific for the FR1of Vκ and reverse primers specific for the FR3 of Vκ. Sense primers (KFR3, K2FR3 and K3FR3) that contained the rabbit LCDR3 sequence flanked by human kappa FR3 and FR4 sequences were designed. Using a phagemid clone containing a human κ-Fab as a template, 5′ truncated κ-pelB fragments were amplified using these primers in combination with the reverse primer lead-B that is specific for the 3′ end of the pelB leader sequence (Table I). The human V κ products amplified from the human bone marrow cDNAs and the κ-pelB fragment were fused by PCR overlap extension using the sense extension primer exthuvl 5′ GCGGAGGAGCTTGCTAGCTGCGAGGGGCCC AGGCGGCCGAGCTC 3′ and the reverse primers leadB. The resulting ˜750 bp PCR products encode human kappa light chains that represent a V κ library derived from bone marrow of 6 human donors combined with the LCDR3 of ST6.

Similarly, Vλ genes were amplified from first strand human bone marrow cDNAs from using sense primers specifc for the FR1, and reverse primers specific for the FR3. A Sense primer, LFR3, that contained the rabbit LCDR3 sequence flanked by human lambda FR3 and FR4 sequences was designed and used with the reverse primer leadB to amplify a 5′ truncated λ-pelB fragment from a phagemid clone containing a human λ-Fab. The human Vλ products and the λ-pelB fragment were fused by PCR overlap extension using the sense extension primer exthuvl and the reverse primers leadB (Table 1 above).

Using the primers leadVH and dpseq (Table 1 above) the ST6-Fd fragment was amplified from template DNA (pPho-ST6 plasmid). The ˜700 bp PCR product was fused to the combined kappa and lambda light chain PCR products by overlap extension using the primers ext (sense, 5′ GCGGAGGAGCTTAGCTGCGAG 3′) and dpex (reverse; Table 1 above). The resulting ˜1500 bp PCR product encoded a human light chains library containing the ST6-LCDR3 linked via the pelB linker sequence with the original chimeric ST6 Fd fragment. The PCR product was SfiI cut and ligated into appropriately cut pComb3X vector DNA.

2.6 Heavy Chain Humanization:

The sequences of the V_{H}-specific oligonucleotides primers used for the humanization of the ST6-heavy chain are given in Table I. Human V_{H} genes were amplified from human bone marrow cDNA obtained from six volunteers using sense primers specific for the FR1 of V_{H} and a reverse primer specific for then 3′ end of FR3 of V_{H}. A sense primer, HFR3, which contained the rabbit HCDR3 sequence flanked by human heavy chain FR3 and FR4 sequences was designed. Using a phagemid clone containing a human Fab as a template, a 5′ truncated V_{H} fragment was amplified from a cloned HC fragment using this primer in combination with the reverse primer HSCG-1234-B (5′ CCTGGCCGGCCTGGCCACTAGTGACCGATGGGCCCTTGGTGGARGC 3′). HSCG-1234-B is specific for the 5′ end of the γ-CH1 sequence. The human V_{H} products amplified from cDNA and the 5′ truncated V_{H} fragment containing the ST6-HCDR3 were then fused by PCR overlap extension using the extension primers HRML-F (sense, 5′ GGTGGTTCCTCTAGATCTTCC 3′) and RSC-B (reverse, 5′ GAGGAGGAGGAGGAGGAGCCTGGCCGGCCTGGCCACTAGTG 3′). The resulting ˜350 bp PCR products represented a V_{H} library derived from bone marrow of 6 human donors combined with the HCDR3 of ST6. Using the sense primers HSCLamb3 (Marasco et al., PNAS, 90: 7889-7893, 1993), HSCLam2 (Yang et al., 1997 supra), HSCK5 (Jung et al., Curr. Opi. Immunol., 11:319-325, 1999); and the J L-specific reverse primers, HSCJlam1236B (5′ GGAAGATCTAGAGGAACCACCGC CTAGGACGGTCASCTTGGTSCC 3′) and HSCJK2B (5′ GGAAGATCTAGAGGAA CCACCTTTGATCTCCAGCTTGGTCCC 3′) the humanized V_{L} regions were PCR amplified. The JL-specific reverse primers and the HFR1-specific sense primers used for the generation of the V_{H} products had a complementary sequence tail that encoded the seven amino acid linker. The ˜350 bp V_{L} PCR products were fused separately to the PCR products encoding the V_{H}-library by overlap extension using the primers ext (sense) and dpex (reverse). The resulting ˜700 bp PCR products encoded the selected human V_{L} sequences containing the ST6-LCDR3 linked via a 7-mer peptide sequence with the human V_{H} library containing the ST6-HCDR3. The four different scFv-PCR products were SfiI cut and ligated into appropriately cut pComb3X vector DNA.

Using the PCR-crossover clone ST6-H2 that was selected from the first heavy chain-humanization library as a template, a V_{H}-fragment was amplified, using the sense primer HC-5-S that is specific for the ST6-HCDR2 and the reverse HSCG1234-B. This PCR product encoded a 5′ truncated V_{H} region with ST6-CDR2 and -CDR3 and with human FR3 and FR4 regions. Using the FR1 specific sense primers and the reverse primer HC4-S, 3′-truncated V_{H} fragments were amplified from human bone marrow cDNA. The HSC4-S is specific for the 3′ end of human HFR2 and has a sequence tail encoding the HCDR2 of ST6. This region was used as an overlap region to fuse the V_{H} fragments amplified from human bone marrow cDNA with the 5′ truncated V_{H} fragment amplified from ST6H2. The resulting PCR product encodes V H sequences, derived from human bone marrow-cDNA combined with the HCDR2 and the HCDR3 of ST6. Data base searches revealed that the amino acid tryptophan in the end of the ST6-HCDR2 (Kabbat position 62) is not found in human HCDR2 in this position. Therefore, the primers HC-5-S and HC-4-S were designed to generate a V_{H} product that encodes serine on this position. V_{L}-PCR-products encoding the selected human V_{L} sequences were pooled and combined with the V_{H} products by overlap extension PCR using the primer-pair ext (sense) and dpex (reverse). The scFv fragments were SfiI cut and cloned into pComb3X.

2.7 Selection Form Phage Displayed Antibody Libraries:

The phage displayed antibody libraries were panned against immobilized CCR5-N-MBP antigen, using 200 ng of protein in 25 μl PBS for coating on 1 well of a Costar # 3690 96-well plate, 0.05% (v/v) Tween 20 in PBS for washing, and 50 μl of 0.1 M glycin-HCl pH 2.2 for elution. The eluted solution was neutralized using 3 μl of 2 M Tris-base. Typically four rounds of panning were performed. The washing steps were increased from 5 in the first round to 8 in the second round and 12 in the third and 14 in the fourth round. After the last round of panning, the phage-pools obtained during the selection and the initial phage-pool were probed for binding to immobilized CCR5-N-MBP and control antigen (CXCR4-N-MBP) by ELISA. Bound phage were detected with an anti-M13 phage conjugate.

2.8 Characterization of Selected Clones:

Twenty to thirty clones derived from the last round of selection were grown to an OD600 nm of ˜0.5, induced with IPTG (2 mM) for 24-30 hours and supernatants from the culture were probed for binding to immobilized CCR5-N-MBP antigen and control antigen (CXCR4-N-MBP) by ELISA. The antibody fragments were detected with anti-human IgG reagents (clones selected from the Fab-library) or with an anti-HA conjugate (scFv-clones). The clones that gave a positive signal by ELISA were further analyzed by DNA fingerprinting. For this, phagemid-DNA was used as a template to amplify Fab or scFv encoding sequences with the flanking primers ompseq (5′-AAGACAGCTATCGCGATTGCAG-3′) and gback (5′-GCCCCCTTATTAGCGT TTGCCATC-3′), and digested with the restriction endonuclease BstO I. Two or more clones representing each fingerprint were further analyzed by DNA sequencing. Using the leader sequence specific primers ompseq and pelseq (5′ CTATTGCCTACGGCAGCCGCTG-3′), the DNA sequence of the V_{L} and V_{H} regions, respectively, was determined for the Fab-clones. The primers ompseq and HRML-F (5′ GGTGGTTCCTCTAGATCTTCC 3′) were used to sequence the V_{L} and V_{H} region of scFv clones.

2.9 Expression of ST6 and ST6/34 as Whole IgG:

The mammalian IgG expression vector PIG-10 (Karlstrom et al., PNAS, 97:3878-3883, 2000) was used to express ST6 and ST6/34 as whole IgG antibodies. Using the sense primers PIG-6-HC (5′ GAGGAGGAGGAGGAGGAGCTCACTCCC AGTCGTTGGAGGAGTCCGGG 3′) and the reverse primers dpseq (Table 1 above), the V_{H} sequence of ST6 was amplified from pComb3X plasmid DNA containing the ST6-Fab insert. Similarly using the sense primer PIG-6/34-HC (5′ GAGGAGGAGGAGGAGGAGCTCACTCCGAGGTGCAGCTGGTGGAGTCT 3′) and the reverse primers dpseq, the V_{H} sequence of ST6/34 was amplified from pComb3X plasmid DNA containing the ST6/34-scFv insert. The PCR products encoding the V_{H} region were cut using the restriction endonucleases SacI, that was introduced through the sense primers and Apa I which is naturally occurring in the 5′ end of the CH1 site and ligated into the appropriately cut PIG-10 vector. The ST6-light chain encoding sequences were amplified from pCombX containing the ST6-Fab fragment using the sense primers PIG-6-LC 5′GCTGCCAGGTGCCAGATGTGC CGAGATCGTGCTGACCCAGACTC 3′ and the reverse primers lead-B (Table 1). The light chain of ST6/34 was amplified from clone pComb3XST6/13A, obtained from the light chain humanization using the sense primer PIG-13-LC 5′ GCTGCCAGGTGCCAGATGTGCCCAGTCTGCCCTGACTCAGCCTC CCTC 3′ and the reverse primers lead-B (Table 1). The light chain products were re-amplified using the sense extension primer PIG-LCext 5′ GAGGAGGAGGAGGAGACTAGTG CTCTGGCTGCCAGGTGCCAGATGT 3′ in conjunction with lead-B. The PCR products encoding the light chain fragments were digested using the restriction endonucleases SpeI introduced through the sense primers and XbaI on the 3′ end of the light chain encoding sequences. The light chain inserts were cloned into the appropriately cut PIG-10 vector DNA containing the corresponding VH regions and the resulting plasmids were named PIG10ST6 and PIG10ST6/34. Upon transfection of PIG10ST6/34 into mammalian cells a human ST6/34IgG1 antibody is produced. PIG10ST6 encodes a chimeric antibody with human IgG1/Kappa constant regions and rabbit VL and VH regions. PIG10ST6/34 and PIG10ST6 plasmid-DNA was used to transiently transfect 293T cells using LipofectAmine according the manufacturers' protocol. Approximately 36 hours after transfection, IgG containing medium was harvested from the cultures and replaced with fresh medium. Culture medium was harvested again after another 36 hours. IgG containing culture supernatants were concentrated and purified by affinity chromatography using a 5-ml Protein G HiTrap column attached to a FPLC system (both from Amersham Pharmacia Biotech). PBS was used for equilibration and washing and bound IgG was eluted with 0.5 M acetic acid. Antibody containing fractions were neutralized using 0.5 volumes 1 M Tris-HCl pH 9.0 and brought into PBS. Purified antibodies were analyzed by SDS-PAGE and stained using Coomassie Blue. Their concentration was estimated using the Bradford assay.

2.10 Epitope Mapping of ST6 (and ST6/34):

A phage peptide library (Ph.D.12, NEB) that consists of filamentous phage displaying random 12-mer peptides via a minor coat protein was panned against ST6. Purified ST6-Fab (6 μg) were coated to ELISA plate wells and binding phage were selected from the library according the manufacturers' protocol. After four rounds of panning, the selected phage pool and single phage clones were tested for binding to ST6-Fab and a control-Fab by ELISA. For that 1 μg of ST6-Fab was coated and bound phage were detected with an anti-M13 phage conjugate (used at 1:2000). Single stranded phage DNA was prepared according the manufacturers protocol and used for DNA sequence analysis to determine the displayed peptide sequence.

2.11 Transfection of 293T Cells (Using Chemokine Receptor Encoding Plasmids and Intrabody Encoding Plasmids.)

The eucaryotic expression plasmid pcDNA3.1.Zeo was modified for intrabody production as described in example 1. Briefly, two SfiI sites were introduced into the multiple cloning site. A human kappa leader sequence was cloned into the vector upstream the 5′ SfiI site. Downstream of the 3′ SfiI site a sequence encoding the HA-tag (YPYDVPDYA) and an ER retention signal (KDEL) followed by a stop codon was introduced. The ST6-scFv-insert was cloned into the modified vector and the resulting plasmid was named pIB6. Using the assymetrical Sfi I sites, the insert encoding ST6/34 scFv was also cloned into the pcDNA3.1.Zeo vector modified for intrabody expression and the resultant plasmid was designated pIB6./34.

293T cells were cotransfected with expression vector plasmid encoding the coreceptors (CCR5 and CXCR4) using LipofectAmine according the manufacturers' protocol. For some experiments, cells were cotransfected with the same amount of plasmid encoding CCR5 specific intrabody (pIB6 or pIB6/34) or with control plasmid - pcDNA 3.1/Zeo containing no insert.

2.12 Flow Cytometric Analysis of Transfected 293T Cells:

For surface staining, cells were incubated with unconjugated primary antibodies for 30 minutes (1 μg/ml), or with PE conjugated primary antibodies (1:100). Cells incubated with unconjugated primary antibody were washed and stained with appropriate FITC or PE conjugates (1:100).

For intracellular staining, cells were permeabilized with phosphate buffered saline (PBS) containing 4% paraformaldehyde and 0.1% saponin (Sigma) for 10 minutes and washed. Cells were then incubated with unconjugated primary antibodies for 30 minutes (1 μg/ml), or with PE conjugated primary antibodies (1:100). Cells incubated with unconjugated primary antibody were washed and stained with appropriate FITC or PE conjugates (1:100). Throughout the intracellular staining, the washing and staining buffers contained saponin (0.1%). After staining and washing, the cells were resuspended in PBS without saponin.

Cells were analyzed on Becton Dickinson Flow cytometers (FACScan, FACSort or FACSCalibur) using Cell Quest software.

3. Results

3.1 Selection of CCR5-specific Antibody Fragments From a Phage Displayed Chimeric Rabbit Fab-library:

A rabbit was repeatedly immunized with a fusion protein containing the N-terminal domain of human CCR5 (CCR5-N-GST). Analysis of the sera by ELISA using a different fusion protein (CCR5-N-MBP) showed a strong immune response to the peptide representing the N-terminal domain of CCR5. The immune sera specifically reacted with cells transfected to express human CCR5 by flow cytometry.

For the generation of a rabbit antibody library displayed on phage, RNA was isolated from bone marrow and spleen of the immune rabbit and was reversely transcribed. V_{L} and V_{H} coding sequences were PCR-amplified from first strand cDNA using a variety of primer combinations designed to amplify most of the known rabbit antibody sequences (Table 1 above).

We used a chimeric Fab format for the construction of the library. The reverse primers used for the amplification of the V_{L} and V_{H} sequences have sequence tails specific for the 5′ end of the human CL and CH1 regions. In a second round of PCR reactions the variable domains of the rabbit light and heavy chains were fused to PCR-fragments encoding the constant human constant domains. The PCR-fragments encoding the chimeric light chains and the chimeric heavy chain Fd-fragment were fused in a final PCR overlap extension step. The PCR product encoding the chimeric Fab library was cloned into the phagemid vector pComb3H (Rader et al., 1997supra) to generate a library of ˜5'107 independent clones.

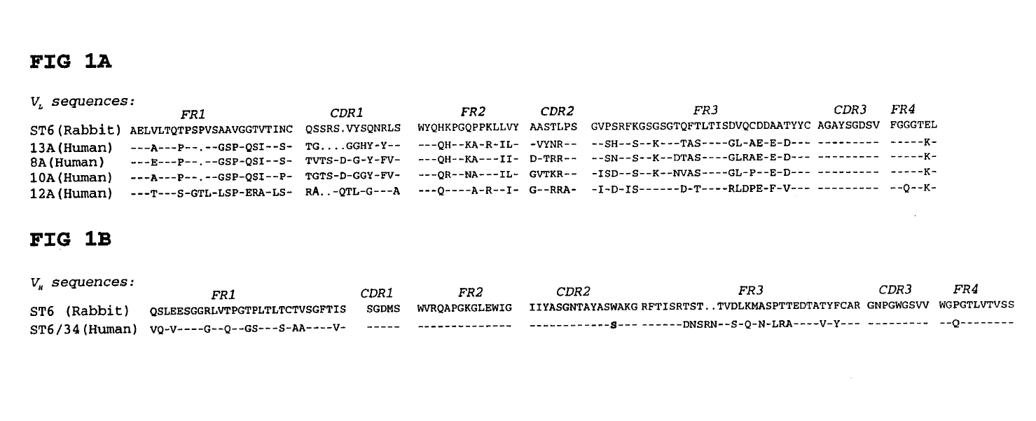

The phage-library displaying chimeric rabbit/human Fab was panned against immobilized CCR5-N-MBP for four rounds. The selected clones that specifically bind to the N-terminal peptide of CCR5 showed little sequence variation and had identical CDR sequences. One clone ST6 that binds strongly to proteins containing the N-terminal peptide of CCR5 and to cells expressing CCR5 was chosen for further characterization (Sequence shown in FIGS. 1A and 1B) and humanization.

3.2 Light Chain Humanization:

Using bone marrow obtained from six healthy donors as a source, V_{L} PCR products that stretched from FR1 to the 3′ end of FR3 were amplified. Sense primers that contained the rabbit-ST6-LCDR3 sequence flanked by human kappa or lambda framework sequences were designed. Using these primers, we generated PCR-fragments from phagemid templates containing human (λ or κ) Fabs that encoded 5′-truncated human light chain and the pel-B leader fragments. Sequence comparison of the LFR4 region was done to chose human λ and κ clones that had a high homology with the ST6 in this region. The V λ and V κ-PCR products that were generated from bone marrow-cDNA and the 5′ truncated λ and κ light chain-pelB-PCR products were fused separately by PCR through overlapping sequences in the LFR3. The resulting human λ and κ light chain fragments, that contained the ST6-LCDR3 sequence were combined with the ST6-Fd encoding fragment by overlap extension PCR. The PCR product was cloned into the phagemid vector pComb3X to create a phage displayed chimeric Fab library with ˜1×10⁸ independent clones. The Library was panned against CCR5-N-MBP and after four rounds of selection, single clones were tested for specific binding and by DNA fingerprint analysis. Four clones ST6/8A, ST6/10A, ST6/12A, ST6/13A, that were different in their sequence and showed strong binding to proteins containing the N-terminal peptide of CCR5, were selected for further humanization and their amino acid sequences are shown in FIGS. 1 (see also sequence listing). ST6/8A, ST6/10A, ST6/13A are human lambda light chains and have V segments of the VL2 family. Clone ST6/12A has a human kappa light chain and its V segment was determined to be of the κIII subgroup. The Fab containing supernatant derived from clone ST6/13A gave a ELISA signal comparable to the ones obtained with ST6-supernatants. The other IPTG-induced supernatants derived form Fab clones with humanized light chains gave weaker signals when probed with CCR5-N-MBP. This might indicate a weaker affinity of these antibodies. In combination with a humanized heavy chain one of these light chains could have a higher affinity than 13A since the interaction of V_{L} and V_{H} (V_{L}/V_{H} interface) is important for the antigen binding. Therefore, the selected humanized light chains ST6/8A, ST6/10A and ST6/12A were also used for the construction of the heavy chain humanization libraries.

3.3 Heavy Chain Humanization:

Using a strategy similar to the one that was employed for the light chain humanization, a human V_{H}-library was constructed, where the heavy chain V_{H} gene sequences derived from human bone marrow were combined with the original ST6-HCDR3. Since the final humanized antibody should initially be used as an intrabody, the heavy chain humanization was carried out in the single chain format.

Using bone marrow obtained from six healthy donors as a source, V_{H} PCR products that stretched from FR1 to the 3′ end of FR3 were amplified. A sense primer that contained the ST6-HCDR3 sequence flanked by human HFR3 and HFR4 sequences was designed and used to generate a PCR-product encoding a 5′-truncated human V_{H}-fragment using a human Fab sequence as a template. For that we chose a human Fab clone with a HFR4 sequence that was highly homologous to the ST6-HFR4. The PCR product was fused to the V_{H}-PCR products generated from human bone marrow cDNA by overlap extension PCR to create a library of human V_{H} sequences with the HCDR3 derived from ST6. Each of the selected humanized ST6-LCDR3-containing V_{L} sequences (8A, 10A, 12A, 13A) was combined separately with the human VH libraries to construct four scFv libraries. The scFv inserts were cloned into the phagemid vector pComb3X. The estimated sizes for the V_{H}-libraries were 1.2×10⁸ (V_{L}-8A), 1.3×10⁸ (V_{L}-10A), 9.1×10⁷ (V_{L}-12A), 7.6×1.0⁷ (V_{L}-13A) independent clones. The libraries were selected against CCR5-N-MBP by four rounds of panning. When analyzed, none of the selected CCR5-N-MBP-binding clones had a completely humanized V_{H} sequence. Most were ST6V_{H}-PCR-contaminants and one clone, ST6-H2 that was isolated from the V_{L}-13A-library was a PCR-crossover products between a human V_{H} clone and the original ST6 V_{H}. The fact that the 5′-end including HCDR2 of the selected crossover-clone ST6-H2 was derived from the ST6-V_{H} and the 3′-end was derived from the human V_{H} library, let us to suspect that the ST6-HCDR2 might play a crucial role in the antigen-binding. Therefore we amplified shorter 5′-V_{H} sequences from human bone marrow-cDNA using a reverse primer specific for the 5′ end of the FR2 (upstream of the human HCDR2). The products encoding human V_{H} sequences were fused to a PCR product amplified from the PCR-crossover-clone ST6-H2. The resulting humanized V_{H} library that contained the CDR2 and CDR3 of ST6 was combined with the four selected light chains to form a new scFv product. A potential immunogenic trypthophan in the grafted ST6-HCDR2 was converted to serine, which is the prevalent amino acid in human VH-sequences in this position. The insert was cloned into pComb3X to generate a library of ˜3.3×107 independent clones. This new V_{H} library panned against CCR5-N-MBP. The selected clones were tested for binding to CCR5-N-MBP and their DNA sequence was determined. One of the selected clones ST6/34 was chosen for further analysis. ST6/34 was strongly binding to the N-terminal peptide of CCR5, had the humanized light chain sequence derived from ST6/13A. The human origin of the selected V_{H} sequences of ST6/34 was confirmed by sequence data base comparison (see SEQ ID NO: 3 and NO:4) and is aligned with the amino acid sequence of the parental antibody ST6 in FIGS. 1.

3.4 Epitope Mapping of ST6:

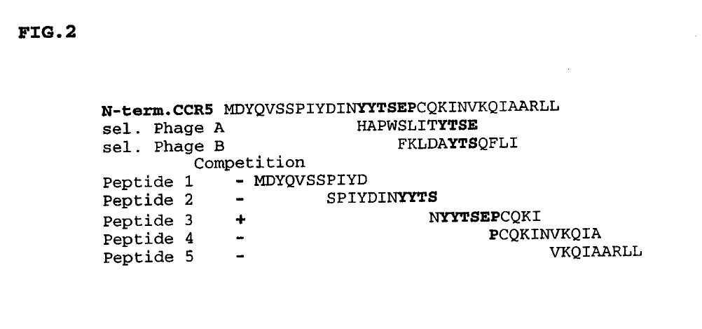

Using a phage displayed peptide library, we selected phage displaying peptides that were specifically bound by ST6 (by ELISA.). Two types of peptides were selected (FIG. 2A). Both selected peptides shared a three amino acid-motiv (YTS) with the N-terminus of CCR5 (amino acids 16-18). A fourth aa (E) was identical with the CCR5 in case of one selected peptide and was similar with the other selected motiv (Q). The specificity of ST6 to the YTSE region was confirmed by using overlapping 11-mer peptides streching the N-terminus of CCR5 to compete with the binding of the selected phage. Peptide CCR5-3 (that streched this region) completely blocked binding of the selected phage to immobilized ST6. Peptide CCR5-2 that contains YTS (on the c-terminus) does not block binding. This is to be expected since the N-terminus of CXCR4 also contains the YTS motive (YTSD) but is not bound by ST6. Several MBP fusion peptides were generated to determine the minimal epitope of ST6. ST6/34-IgG was also probed with the same fusion proteins to confirm that the epitope-specificity was retained in the humanization process. ST6-IgG and ST6/34-IgG but not B12-IgG, which was used as a negative control antibody, bound a fusion protein, which stretched a 6 amino acid motive (aa 14-19, YYTSEP) of the N-terminal extra-cellular domain of CCR5. ST6-IgG and ST6/34-IgG did not bind the other fusion proteins tested. Judged from the ELISA signal, the binding of ST6/34-IgG to the MBP-YYTSEP-fusion protein was slightly weaker compared to the ST6-IgG. ST6-IgG and ST6/34-IgG both bound equally strong to MBP-N-CCR5, which was included as a positive control in the experiment

3.5 The Humanized Intrabody ST6/34 Blocks CCR5 Expression as Efficiently as the Parental Rabbit Antibody ST6.

We showed in example 1, that the ST6 efficiently blocks the expression of CCR5 when expressed as an intrabody with an ER retention signal. The humanized version of ST6, ST6/34 was cloned into an intrabody expression vector described in example 1, and the resulting construct was named pIB6/34. To study the effect of the intrabody, cotransfections were performed, using the same amount of chemokine receptor expression plasmid and expression plasmids encoding ST6 (pIB6) or ST6/34 (pIB6/34).

Upon cotransfection with pIB6-DNA or pIB6/34-DNA the surface expression of human CCR5 was greatly reduced. The percentages for positive staining are: 66% -pcDNA; 5.9% - pIB6; 2.7% - pIB6/34 and 2.1% for the staining with the irrelevant control antibody. Cotransfection with pIB6/34-DNA let to a slightly stronger reduction of CCR5 expression. Cells derived from the same experiment were also permeabilized and stained for intrabody expression using an antibody specific for the HA-tag sequence. This staining indicated higher amount of intrabody ST6 in the cells. The stronger reduction of CCR5 expression upon cotransfection with pIB6/34 does not appear to be due to better expression of humanized intrabody ST6/34 in the cells. A Donkey anti-rabbit-IgG antibody reacts with the rabbit ST6 intrabody but not with the humanized ST6/34 when used for intracellular staining. The effects of pIB6 and pIB6/34 are specific for CCR5, since they did not affect the expression of CXCR4 when cotransfected with CXCR4-expression plasmid. The percentages for positive staining are: 74% - pcDNA; 77.6% - pIB6; 78.4 - pIB6/34 and 2.1% for the staining with the irrelevant control antibody.

3.6 ST6/34IgG and ST6IgG Binds to Cells Expressing CCR5 as Shown by Flow Cytometry.

DNA-fragments encoding the light chains and the V_{H}-sequences of ST6 and ST6/34 were cloned into a whole IgG expression vector (PIG10) that encodes the CH1-3 of human IgG1. The resulting plasmids PIG10ST6 and PIG10ST6/34 were used to transiently transfect 293T cells. Whole IgG was purified from the culture supernatants using protein G affinity chromatography. The integrety of the purified product was confirmed be SDS-gel electrophoresis. Whole IgG was used to stain 293T cells transfected to express human CCR5 for flow cytometry. The chimeric rabbit/human ST6IgG as well as the human ST6/34-IgG bound strongly to cells transiently transfected to express human CCR5. No binding was obtained with cells transiently transfected to express human CXCR4.

### EXAMPLE 3

### CXCR4 Intrabodies

In order to select CXCR4-specific antibody fragments from a phage displayed chimeric rabbit Fab-library, fusion protein of Example 2 (Methods part) containing the N-terminal extracellular domain (amino acid 1-33) of CXCR4/Fusin is generated in a similar manner and is called CXCR4-N-GST and CXCR4-N-MBP. A rabbit is then repeatedly immunized with a fusion protein containing the N-terminal domain of human CCR5 (CXCR4-N-GST). Analysis of the sera by ELISA using a different fusion protein (CXCR4-N-MBP) shows a strong immune response to the peptide representing the N-terminal domain of CCR5. The immune sera specifically reacts with cells transfected to express human CXCR4 by flow cytometry.

According to protocols described in Example 2, for the generation of a rabbit antibody library displayed on phage, RNA is isolated from bone marrow and spleen of the immune rabbit and is reversely transcribed. V_{L} and V_{H} coding sequences are PCR-amplified from first strand cDNA using a variety of primer combinations designed to amplify most of the known rabbit antibody sequences. Chimeric Fab format for the construction of the library is suitable. The reverse primers to be used for the amplification of the V_{L} and V_{H} sequences have sequence tails specific for the 5′ end of the human CL and CH1 regions. In a second round of PCR reactions the variable domains of the rabbit light and heavy chains are fused to PCR-fragments encoding the constant human constant domains. The PCR-fragments encoding the chimeric light chains and the chimeric heavy chain Fd-fragment are fused in a final PCR overlap extension step. The PCR product encoding the chimeric Fab library is cloned into the phagemid vector pComb3H (Rader et al., 1997 supra) to generate a library of at least 10E7 independent clones.

According to protocols described in Example 2, the phage-library displaying chimeric rabbit/human Fab is panned against immobilized CXCR4-N-MBP for four rounds. Several clones that bind strongly to proteins containing the N-terminal peptide of CXCR4 and to cells expressing CXCR4 are selected.

### EXAMPLE 4

### In vivo Expressions of CCR5 and CXCR4 Intrabodies

The cellular entry of HIV is mediated by the specific interaction of viral envelope glycoproteins with the cell-surface marker CD4 and a chemokine receptor (CCR5 or CXCR4). To assess the effect of an anti-CCR5 or anti-CXCR4 intrabody (“intrabody” herein under) on macrophage differentiation, CD34+ hematopoietic progenitor cells were transduced with a retroviral vector carrying anti-CCR5 or anti-CXCR4 intrabody and allowed to differentiate in the presence of appropriate cytokines. Intrabody-transduced CD34+ cells can differentiate normally into mature macrophages that carried CD14 and CD4 surface markers, expressed the anti-CCR5 or anti-CXCR4 intrabody, and can show significant resistance to viral infection upon challenge with the HV-1 BaL strain (see previous examples 1 and 2). Using an in vivo thymopoiesis model, the effect of anti-CCR5 or anti-CXCR4 intrabody on stem cell differentiation into thymocytes can be evaluated by reconstituting SCID-hu mice thymic grafts with intrabody-transduced CD34+ cells. FACS analysis of cell biopsies at 4 and 6 weeks postengraftment for HLA, CD4, and CD8 markers can show comparable levels of reconstitution and similar percentages of subpopulations of thymocytes between grafts receiving intrabody-transduced and control CD34+ cells. RT-PCR assays can demonstrates the expression of the intrabody in CD4+, CD8+, and CD4+/CD8+ thymocyte subsets derived from intrabody-transduced CD34+ cells. These results can indicate that anti-CCR5 and anti-CXCR4 intrabodies can be introduced into hematopoietic stem cells without adverse effects on their subsequent lineage-specific differentiation and maturation. The expression of intrabodies in HIV-1 target cells offers a novel gene therapy strategy to control HIV infection.

After the macrophages grow out from the intrabody-transfected stem cells, the animals can be challenged with r5 virus to study the effect as in Gauduin et al. (Nat. Med., 3:1389-93, 1997). In this model, severe combined immunodeficient (SCID) mice are populated with human intrabody-transfected peripheral blood mononuclear cells (PBMCs as described above) and infected with HIV-1. We can find that the potent neutralizing human intrabodies ST6 and ST6/34 is able to completely protect even when given several hours after viral challenge. The results are encouraging for antibody-based postexposure prophylaxis and support the notion that antibody induction could contribute to an effective vaccine.

Further 5 Vaccine Containing CCR5-like Peptides

A vaccine is prepared as a hand-made emulsion of squalene:mannide oleate vehicle in a ratio of 4:1 (v:v) formulated with a CCR5-peptide-diphtheria toxoid conjugate (25 molecules synthetic CCR5 peptide/105 Da of DT) to nor-muramyl dipeptide adjuvant ratio of 20:1 (w:w) dissolved in sterile saline. CCR5-peptides contain 24 amino acids of the following sequence YTSEYTSE YTSE YTSQYTSQYTSQ. The inoculation volume is 0.4 ml for 0.5 mg dose, 0.8 ml for 1.0 mg dose and 1.6 ml for a 2.0 mg dose (based upon conjugate weight). Patients can be enrolled into a low dose regimen which consisted of 0.5 mg vaccine on day 0, day 28, day 70 and week 16. Other patients in a high dose regimen can receive 2.0 mg of vaccine on day 0 followed by 1.0 mg on day 28, day 70 and week 16. The vaccine is to be administered intramuscularly. A standard solid phase, indirect enzyme-linked inummosorbent assay (ELISA) is used for analysis of patient antisera for anti-DT antibodies. Patient antisera with sufficient anti-CCR5 antibody titer is assayed for binding to CCR5-N-MBP (see example 2) using a competition ELISA (Chang S P et al., J Immunol., 128: 702-705, 1982). Prior to the competition ELISA, optimal antigen coating concentration and optimal antiserum dilution for the competition ELISA are determined for each antiserum by indirect ELISA. The immune response to CCR5 is to be measured in the serum of patients from 0 to 24 weeks post initiation of vaccination. Significant anti-CCR5 antibody production can be observed by week 12 of this vaccination protocol (data not disclosed yet).

### EXAMPLE 6

### CCR5 Antiidiotypic Antibodies

Phage libraries can be produced using the following protocol. Male rabbits are subcutaneously (s.c.) injected on days 0 and 15 with 50 Pg of ST6 scFv (see example 1) in 0.2 ml of complete Freund's adjuvant (Difco, Italy) and on days 21 and 28 with the same dose of antigen in 0.2 ml of incomplete Freund's adjuvant (Difco). A final booster injection is given intraperitoneally on day 35, and three days later the rabbits are sacrificed and their spleens removed. Spleen cells are resuspended in tissue culture medium and dispensed into 75 cm2 tissue culture flasks previously coated with scFv ST6 in carbonate buffer (pH 9.6) and blocked with 2% non-fat dry milk (Sigma, Italy). After overnight incubation at 37C in 5% C02, non-adherent spleen cells are removed and mRNA is extracted from adherent cells directly in the panning flask using guanidium isothiocyanate. After purification by affinity chromatography on oligo(dT)- cellulose (QuickPrep mRNA purification kit, Pharmacia), reverse-transcription of the purified mRNA is performed with a murine reverse transcriptase by priming with random hexadeoxyribonucleotides. To clone and express antibody fragments, a commercial system (Recombinant Phage Antibody Sys., Pharmacia) is used. The heavy and light chain antibody genes are amplified in two separate PCR reactions, by using two sets of specific primers. The heavy and light chain DNA products are assembled into a single gene using a DNA fragment encoding a (GIY4Ser)3 linker, acting as a bridge between the carboxy-terminus of the VH chain and the amino-terminus of the VL chain. The gene is then reamplified to introduce two restriction sites (Sfi IlNot 1) for cloning into a specific phagemid vector (pCANTAB 5E). The ligated vector, containing the scFv genes linked to a sequence encoding for a C- terminal 13 amino acid peptide tag (E-tag) and followed by an amber translation stop codon, is introduced into a competent supE E. coli strain (TGI) which was then infected with the M13KO7 helper phage to yield recombinant phages displaying recombinant scFv antibodies on their tips.

Phage selection can be performed using the following protocol. Recombinant phages are dispensed to a 25 cm2 tissue culture flask that had been previously coated with 5 ml of scFv ST6 in carbonate buffer (pH 9.6). After an incubation of 2 hours at 37C and extensive washing, log-phase TGI cells are added to the flask and incubated at 37′C. for I hour. The suspension is transferred into sterile tubes and, after the addition of ampicillin (100 ˜mg/ml), glucose (2%) and M13KO7 helper phage, and further incubated with shaking at 37C. After production of the recombinant phage supernatant, a second round of panning is repeated and the cells plated onto ampicillin-containing agar plates. Recombinant phage supernatants, obtained from the master plates are screened in the wells of microtiter plates previously coated with scFv ST6. Bound phages can be detected using peroxidase-conjugated sheep anti-M13 phage antibodies

After two pannings of the library, several different phage clones produced strong reactions in ELISA tests using again wells coated with scFv ST6. Further analysis show (data not disclosed yet) that some clones actually produce scFv molecules recognizing an idiotypic determinant of scFv ST6 (called “CCR5-antiiodiotypic scFv” hereinunder). In further experiments in which mice are immunised with selected CCR5-antiiodiotypic scFv, the anti-CCR5 immune response generated against these molecules is confirmed (data not disclosed yet).

What is claimed is:

1. A method for inhibiting, diminishing, preventing or treating pathogenic infection of cells comprising expressing a recombinant antibody protein fused to an intracellular anchor means, wherein said antibody is specific for a surface receptor of said cells necessary for pathogenic infection.

2. The method of claim 1, wherein said antibody is selected from CCR5 and CXCR4 specific antibodies.

3. The method of claim 2, wherein said antibody is a scFv-fusion protein comprising a scFv domain that immunoreacts with CCR5 or CXCR4 fused to an intracellular anchor means.

1 The method of claim 3 wherein said scFv-fusion protein comprises amino acid residues selected from SEQ ID NO:1 to NO: 4.

4. The method of claim 1 wherein said intracellular anchor means is an endoplasmic reticulum (ER) retention peptide domain.

5. The method of claim S wherein said ER retention peptide is KDEL.

6. The method of claim 1 wherein said expression comprises in vivo or ex vivo transformation of CCR5- or CXCR4- bearing cell.

7. The method of claim 7, wherein stem cells are transformed, more specifically hematopoietic cells.

8. The method of any of the preceding claims, wherein CCR5 and CXCR4 specific antibodies are co-expressed in cells.

9. The method of any of the preceding claims 1-8, wherein said antibody is humanized.

10. A recombinant antibody protein fused to an intracellular anchor means which is specific for a surface receptor necessary for pathogenic infection.

11. The antibody of claim 11, wherein said antibody is selected from CCR5 and CXCR4 specific antibodies.

12. The antibody of claim 12, wherein said antibody is a scFv-fusion protein comprising a scFv domain that immunoreacts with CCR5 or CXCR4 fused to an intracellular anchor mean.

13. The antibody of claim 13, wherein said scFv-fusion protein comprises amino acid residues selected from SEQ ID NO:1 to NO: 4.

14. The antibody of claim 11, wherein said intracellular anchor mean is an endoplasmic reticulum (ER) retention peptide domain.

15. The antibody of claim 15 wherein said ER retention peptide is KDEL.

16. The antibody of any of the preceding claims 11-14, wherein said antibody is humanized.

17. A recombinant antibody that immunoreacts with CCR5 or CXCR4.

18. The antibody of claim 18 wherein said antibody is humanized.

19. The antibody of claim 18 wherein said antibody is a single chain antibody (scFv).

20. The antibody of claim 1 wherein said antibody comprises amino acid residues selected from SEQ ID NO:1 to NO:4.

21. A polynucleotide that encodes an antibody according to any of the preceding claims 11 to 20.

22. A viral expression system encoding a polynucleotide of claim 22.

23. Peptides comprising at least YTSE or YTSQ sequence for use in a vaccine or an immunogenic composition intended to control, prevent, diminish or treat HIV infections.

24. An antiidiotypic antibody mimicking CCR5 or CXCR4 epitopes raised from anti-CCR5 and anti-CXCR4 antibodies.
